(19) United States
(12) Patent Application Publication
(10) Pub. No.: US 2019/0083679 A1
(43) Pub. Date: Mar. 21, 2019

(54) Amorphous Inorganic Polyphosphate-Calcium-Phosphate And Carbonate Particles As Morphogenetically Active Coatings and Scaffolds

(71) Applicant: NanotecMARIN GmbH, Mainz (DE)

(72) Inventor: Werner Ernst Ludwig Georg MÜLLER, Mainz (DE)

(21) Appl. No.: 15/527,520

(22) PCT Filed: Nov. 10, 2015
(86) PCT No.: PCT/EP2015/076172

§ 371 Date: May 17, 2017

(30) Foreign Application Priority Data
Nov. 17, 2014 (GB) 1420363.2
Jul. 23, 2015 (GB) 1513011.5
Sep. 2, 2015 (GB) 1515515.3
Oct. 20, 2015 (GB) 1518575.4

(51) Int. Cl. A61L 27/32

(52) U.S. Cl. CPC A61L 27/32; A61L 2300/112; A61L 2420/02; A61L 2430/02; A61L 2300/412

(57) ABSTRACT

This invention concerns a method for the production of amorphous, nano- or microparticular materials based on inorganic polyphosphate (polyP) and calcium phosphate or calcium carbonate that show osteogenic activity. In one aspect of the invention, the inventor shows that amorphous calcium polyphosphate (Ca-polyP) microparticles can be used for biological functionalization of titanium alloy surfaces. The inventive method allows the fabrication of a durable and stable, almost homogeneous and morphogenetically active Ca-polyP layer on titanium oxidized Ti-6Al-4V scaffolds that supports the growth and enhances the functional activity of bone cells, in contrast to biologically inert non-modified titanium surfaces. A preferred aspect relates to the formation of amorphous calcium phosphate (CaP) particles in the presence of low concentrations of sodium polyP. This material causes a strong upregulation of the expression of proteins involved in bone formation. A further aspect of the invention concerns a material containing polyP-stabilized ACC and small amounts of vaterite that exhibits osteogenic activity and supports the regeneration of the implant region in animal experiments. The amorphous materials according to this invention have the potential to be used for bone implants.

This invention concerns a method for the production of amorphous, nano- or microparticular materials based on inorganic polyphosphate (polyP) and calcium phosphate or calcium carbonate that show osteogenic activity. In one aspect of the invention, the inventor shows that amorphous calcium polyphosphate (Ca-polyP) microparticles can be used for biological functionalization of titanium alloy surfaces. The inventive method allows the fabrication of a durable and stable, almost homogeneous and morphogenetically active Ca-polyP layer on titanium oxidized Ti-6Al-4V scaffolds that supports the growth and enhances the functional activity of bone cells, in contrast to biologically inert non-modified titanium surfaces. A preferred aspect relates to the formation of amorphous calcium phosphate (CaP) particles in the presence of low concentrations of sodium polyP. This material causes a strong upregulation of the expression of proteins involved in bone formation. A further aspect of the invention concerns a material containing polyP-stabilized ACC and small amounts of vaterite that exhibits osteogenic activity and supports the regeneration of the implant region in animal experiments. The amorphous materials according to this invention have the potential to be used for bone implants.

### BACKGROUND OF THE INVENTION

The basic building blocks of bone comprise, besides of collagen and water, carbonated apatite [Ca₅(PO₄,CO₃)₃(OH)], as well as hydroxyapatite (HA). The crystalline minerals are likely to be formed from amorphous calcium phosphate (ACP) (Wang Y, et al. (2013) Water-mediated structuring of bone apatite. Nat Mater 12:1144-1153).

Recent evidences suggest that amorphous calcium carbonate (ACC) acts as bioseed for the formation of ACP and carbonated apatite, a material that is formed by carbonic anhydrase(s) (CA), very likely by the soluble CA-II isoform and/or the cell-membrane-associated CA-IX (Wang X H, et al. (2014) Modulation of the initial mineralization process of SaOS-2 cells by carbonic anhydrase activators and polyphosphate. Calcif Tissue Int 94:495-509; Müller W E G, et al. Mineralization of bone-related SaOS-2 cells under physiological hypoxic conditions. FEBS J. 2015 Oct. 10).

ACC is the least stable polymorph of calcium carbonate, which exists both in amorphous and crystalline phases; among the three major crystalline polymorphs, vaterite, aragonite, and calcite, the metastable vaterite is the thermodynamically least stable form of crystalline CaCO₃ (Meldrum F C, Cölfen H (2008) Controlling mineral morphologies and structures in biological and synthetic systems. Chem Rev 108:4332-4432).

Accordingly, bone HA formation can be subdivided into the following three mechanically distinct phases:

1. Enzymatic formation of ACC bioseeds via carbonic anhydrase(s);

2. Non-enzymatic exchange of carbonate ions by phosphate under formation of ACP; and

3. Transition of ACP to the crystalline phase carbonated apatite/HA.

The application of ACC as a potential regeneration-inducing/supporting material has been hampered by the fact that ACC, as such, is not stable. Stabilization of ACC in vivo is regulated by specialized proteins, often in combination with Mg²⁺, while under in vitro conditions non-biogenic additives, like soluble polycarboxylates, again Mg²⁺, triphosphate, or polyphosphonate species freeze ACC to a relative stable phase (Kellermeier M, et al. (2010) Stabilization of amorphous calcium carbonate in inorganic silica-rich environments. J Am Chem Soc 132:17859-17866).

In contrast, vaterite is stable enough to allow dissociation and in turn might act as a potential ion buffering system for bone regeneration and by that could modify transformation processes from CaCO₃ to HA (Schröder R, et al. (2015) Transformation of vaterite nanoparticles to hydroxycarbonate apatite in a hydrogel scaffold: Relevance to bone formation. J Mater Chem B 3:7079-7089).

Recently, the present inventor succeeded to prepare amorphous polyphosphate (polyP)-based microparticles (Müller W E G, et al. A new polyphosphate calcium material with morphogenetic activity. Materials Letters 2015; 148:163-166; Müller W E G, et al. Retinol encapsulated into amorphous Ca²⁺ polyphosphate nanospheres acts synergistically in MC3T3-E1 cells. Eur J Pharm Biopharm 2015; 93:214-223) that act not only in an anabolic way on the osteoblast system, but also provide metabolic fuel for their target cells, like the osteoblasts, and cause an elevation of the intracellular level of ATP as well as an increase in the number of mitochondria (Müller W E G, et al. Amorphous Ca²⁺ polyphosphate nanoparticles regulate the ATP level in bone-like SaOS-2 cells. J Cell Sci 2015; 128:2202-2207. The present inventor further disclosed this biocompatible, biodegradable and biologically active polyP-based material in GB1420363.2. The size of the microparticles described in GB1420363.2 can be adjusted by a defined Pᵢ:Ca²⁺ molar ratio (Müller W E G, et al. (2015) A new polyphosphate calcium material with morphogenetic activity. Materials Lett 148:163-166). The particles formed retained the amorphous state and hence are prone to enzymatic hydrolysis by alkaline phosphatase (ALP).

PolyP is present in considerable amounts in the blood and in larger extent in blood platelets and has been implicated as a phosphate source for the formation of the bone calcium phosphate deposits. From this polymer ortho-phosphate is enzymatically removed via the ALP which might serve as donor for bone mineralization. Previously the inventor described that polyP regulates cell-specific differentiation processes, like the formation of mineral depositions onto bone-forming osteoblasts with the model cell line SaOS-2 cells and the induction of the ALP and shifts the OPG (osteoprotegerin):RANKL (receptor activator of nuclear factor KB ligand) ratio towards anabolic, osteoblast pathway and by that inhibits the function of osteoclasts, using the model cell line RAW 264.7. In addition, polyP induces the genes encoding for the bone morphogenetic protein-2 (BMP-2) and the scaffold structural filamentous system, the collagens. The present state-of-the-art in enzyme-mediated bone formation and the role of polyP has been described in, for example:
Wang X H, Schröder H C and Müller W E G. Enzymatically synthesized inorganic polymers as morphogenetically active bone scaffolds: application in regenerative medicine. Int Rev Cell Mol Biol 2014; 313:27-77
Wang X H, et al. (2015) Polyphosphate as a metabolic fuel in Metazoa: A foundational breakthrough invention for biomedical applications. Biotechnol J. doi: 10.1002/biot.201500168;
Müller W E G, et al. (2015) Non-enzymatic transformation of amorphous CaCO₃ into calcium phosphate mineral after exposure to sodium phosphate in vitro: Implications for in vivo hydroxyapatite bone formation. ChemBioChem 16:1323-1332; and
Müller W E G, et al. Polyphosphate: a morphogenetically active implant material serving as metabolic fuel for bone regeneration. Macromolec Biosci 2015; 15:1182-1197.

There is an urgent need for new bone implant materials because of the limitations of current materials (e.g. long immobilization periods of patients, infections caused by the implant etc). Ideally these implants have to follow the principles of the natural process of bone formation, allowing a fast regeneration of the damaged bone tissue.

In one aspect of the present invention, the inventor describes that polyP can stabilize the ACC phase. In the inventive procedure, at a level of 5% [w/w], polyP considerably suppresses the transformation of ACC to crystalline CaCO₃ and at a percentage of 10% [w/w] the polymer almost completely blocks this process. This finding was unexpected. Previously it has been reported that soluble Na-polyP, spiked with defined molar ratios of Ca²⁺, can be processed to solid nanoscaled nano-/microparticles that remain amorphous (Müller W E G, et al. (2015) A new polyphosphate calcium material with morphogenetic activity. Materials Letters 148:163-166; Müller W E G, et al. (2015) Retinol encapsulated into amorphous Ca²⁺ polyphosphate nanospheres acts synergistically in MC3T3-E1 cells. Eur J Pharm Biopharm 93:214-223). It could not be expected that Ca-polyP can act as a stabilizer for metastable ACC; see also: GB1420363.2; and GB1502116.5.

PolyP acts as a morphogenetically active inorganic molecule on bone cells and induces their mineralization (GB1420363.2, GB1502116.5, GB1403899.6). In the present application, the inventor additionally shows that CaCO₃, containing 5 or 10% [w/w] of polyP, comprises osteogenic potential in SaOS-2 cells as well as in human mesenchymal stem cells (MSC) by inducing ALP and bone morphogenic protein 2 (BMP2) gene expression. Even more surprisingly and unexpectedly, ACC enhanced the stimulatory effect of polyP on BMP2 expression in a “synergistic” way. Moreover, the inventor demonstrates that the inventive ACC/polyP hybrid material is biocompatible and supports regeneration in vivo, making it to a promising scaffold material for bone replacement/implants.

In a further aspect of this invention, the inventors used a technology for fabrication of CaP, starting from calcium chloride and dibasic ammonium phosphate. In a novel approach, besides of the preparation of HA with the characteristic Ca/P molar ratio of 10:6, they prepared CaP mixed with various amounts of polyP. Unexpectedly the inventor found that CaP preparations that contained >10% by weight of polyP (with respect to the modified CaP/HA deposits) are amorphous while the CaP/HA samples that contained <10% by weight of polyP consist of a crystalline phase. All samples were found to support the growth of bone cell-related SaOS-2 cells but, surprisingly, only the CaP preparation, containing 10 weight percent (wt. %) of polyP, elicits strong morphogenetic activity, as determined by measuring the expression of the genes encoding for ALP and collagen type I (marker genes for differentiation of bone and bone-related cells). Based on these results the inventive polyP/CaP-based material might be beneficial for application as bone substitute implant.

Previously, the inventors developed a polyP-based material that is produced at ambient conditions in the presence of a defined concentration of CaCl₂. This material consists of spherical, amorphous particles that are biocompatible and biodegradable.

Now the inventors surprisingly found that this biologically active material, prepared with a size in the microparticulate range, can be used for surface coating of Ti-6Al-4V scaffolds via formation of Ca²⁺ ion bridges to the silane coupling agent APTMS, as demonstrated by electron microscopically and element analytical (EDX) methods. This finding and the high durability and stability of the coating were unexpected, in particular because of the microparticulate nature of the Ca-polyP particles.

Another surprising property of the Ca-polyP coated titanium alloy is its property to act as a suitable matrix for the growth of bone-like SaOS-2 cells despite its markedly reduced surface roughness that should not support cell attachment, and—even more—its ability to induce these cells, in contrast to the untreated titanium scaffolds, to express the key enzymes, carbonic anhydrase IX (CA IX) and ALP, which are involved in the initiation of enzyme-induced bone mineral deposition.

Based on their properties, the inventive coated titanium scaffolds are promising material for the fabrication of high-precision implants with innovative regeneration-eliciting characteristics, which can be produced in an individualized size and shape.

It is well known that Na-polyP is readily chelating Ca²⁺ and forms insoluble precipitates. Therefore it can be expected that addition of Na-polyP to CaCl₂ and (NH₄)₂HPO₄ will result in a co-precipitation of amorphous Ca-polyP and crystalline CaP (HA) mineral deposits.

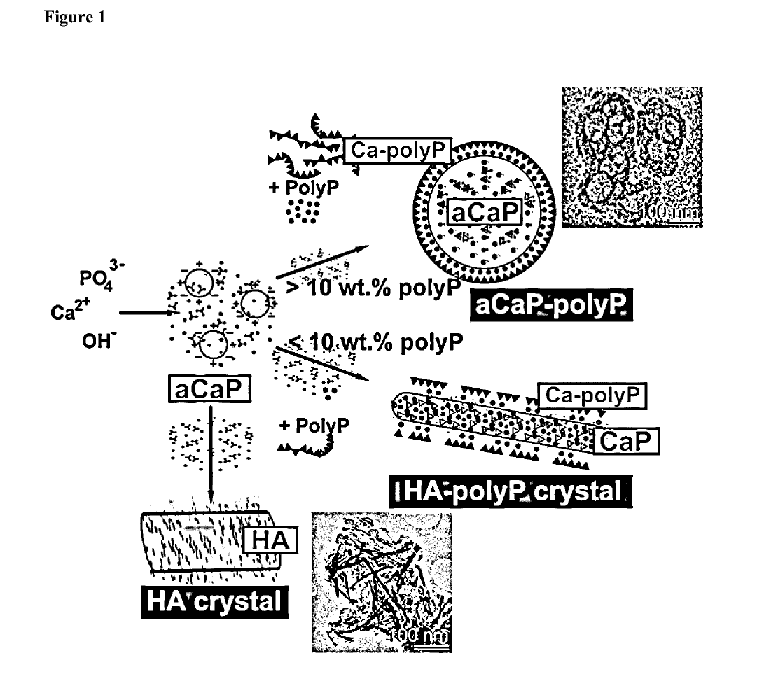

In a further aspect of this invention, the inventors added increasing concentrations of Na-polyP together with CaCl₂ and (NH₄)₂HPO₄, the substrates for HA formation in aqueous solution, during the precipitation procedure. Surprisingly, and unexpected, they found that a content of 10 wt. % polyP prevents the formation of crystalline HA under simultaneous fabrication of amorphous polyP/CaP hybrid particles with a size of around 100 nm. A summary of the results underlying this aspect of the invention is shown in FIG. 1.

This finding is important because the application of crystalline HA, even though being biocompatible, is currently limited to powders, coatings and porous bodies, and non-load-bearing implants due to the adverse physical (low solubility) and mechanical properties (brittleness) (Wang M C, et al. Crystalline size, microstructure and biocompatibility of hydroxyapatite nanopowders by hydrolysis of calcium hydrogen phosphate de hydrate (DCPD). Ceramics Intern 2015; 41:2999-3008). These disadvantages can be circumvented if CaP could be synthesized in an amorphous phase.

The inventor demonstrates that the amorphous polyP/CaP particles (abbreviated: aCaP-polyP) show, in combination with cell growth promoting activity a distinct morphogenetic activity. They found that aCaP-polyP causes a strong upregulation of the two marker genes for bone formation, collagen type I and ALP. The potency of aCaP-polyP is comparable to Ca-polyP.

Based on their properties to elicit morphogenetic activity, the inventive aCaP-polyP particles offer a promising material to be used as artificial bone implant, fabricated from physiological metabolites/polymers.

In a further aspect of this invention, the inventor surprisingly found that the metastable ACC phase can be stabilized by polyP. In human bone, ACC is formed as a precursor of the crystalline carbonated apatite/HA. PolyP is used as a phosphate source for the non-enzymatic carbonate/phosphate exchange. The inventor demonstrates that polyP suppresses the transformation of ACC into crystalline CaCO₃ at a percentage of 5% [w/w] (termed “CCP5”) with respect to CaCO₃ and almost completely at 10% [w/w] (termed “CCP10”). They show that both preparations are amorphous, but also contain small amounts of vaterite, as revealed by XRD, FTIR and SEM analyses.

The inventor demonstrates that the ACC/polyP particles according to this invention exhibit osteogenic activity, in contrast to calcite. They induce the expression of the gene encoding for ALP in SaOS-2 cells as well as in human mesenchymal stem cells (MSC), as well as the expression of BMP2 gene. Furthermore, the inventors demonstrate, in in vivo studies in rats, using PLGA microspheres containing the inventive ACC/polyP material and inserted in the muscles of the back of the animals, that the encapsulated ACC/polyP particles are not only biocompatible but also support the regeneration of the implant region. It is surprising that ACC containing small amounts of vaterite has osteogenic potential and superior properties compared to a biologically inert calcite. Based on these properties the inventive material represents a promising scaffold material for bone implants.

The following patent applications on polyP are deemed relevant: GB1406840.7, GB1403899.6; WO 2012/010520; GB1420363.2; GB1502116.5, and GB1510772.5.

In GB1420363.2 the inventors disclosed a method for producing a material consisting of calcium-polyP microparticles, that shows the following properties: (i) it is amorphous and (ii) it is biologically active in cell systems able to mineralize.

The results have also been reported in: Müller W E G, et al. A new polyphosphate calcium material with morphogenetic activity. Materials Letters 2015; 148:163-166; Müller W E G, et al. Retinol encapsulated into amorphous Ca²⁺ polyphosphate nanospheres acts synergistically in MC3T3-E1 cells. Eur J Pharm Biopharm 2015; 93:214-223; and Müller W E G, et al. Polyphosphate: a morphogenetically active implant material serving as metabolic fuel for bone regeneration. Macromolec Biosci 2015; 15:1182-1197.

The properties of the material described in GB1420363.2 are superior to HA (see also under Examples) and to those of conventional polyP preparations for bone regeneration and repair, e.g., GB1406840.7; and GB1403899.6.

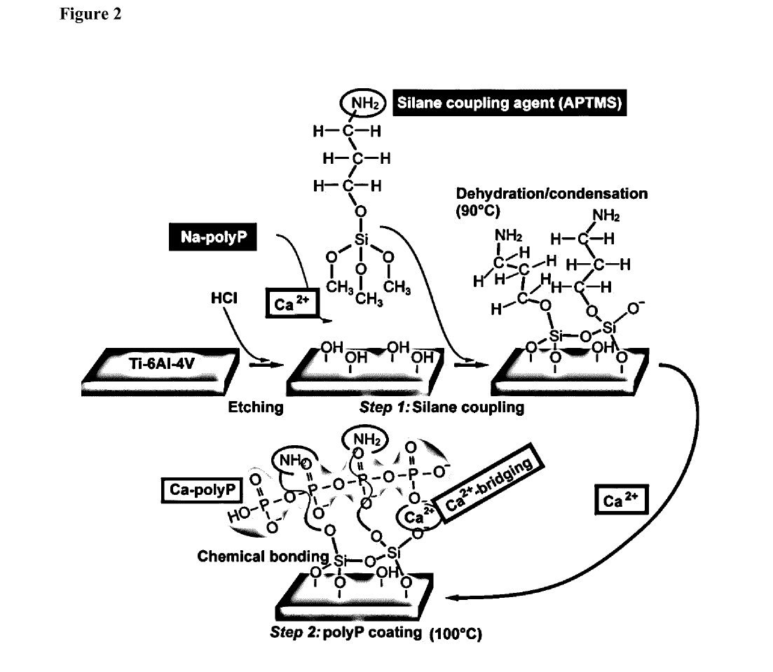

Now the inventors succeeded to develop a procedure through which titanium/titanium alloy can be tightly overlaid with polyP. After etching with HCl the metal surface is covalently linked with APTMS, after which the Ca-polyP particles can attach to the surface via Ca²⁺ ionic linkages (FIG. 2).

The inventive polyP coat at the surface of the metal turned out to be durable and surprisingly stable.

APTMS can be replaced by other silane coupling agents such as, for example, 3-(trimethoxysilyl)propyl methacrylate. The further functional group of APTMS allows the binding of peptides to the silane-coated titanium surfaces, in addition to polyP.

In contrast to the considerably high surface roughness of the untreated titanium discs (approximately 7 μm in maximum), Ti—Ca-polyP discs are smooth with a maximal roughness of 0.8 μm. Usually the degree of cell attachment to very smooth surfaces is lower, compared to moderately rougher surfaces (e.g. Huang H H, et al. Effect of surface roughness of ground titanium on initial cell adhesion. Biomol Eng 2004; 21:93-97). Therefore, it came unexpected that the polyP-coated discs allow SaOS-2 cells to grow with a rate, seen in control assays without any discs.

The inventor shows that the cells in the assays, which contained untreated titanium discs die off after an incubation period of 2 d. This is very much in contrast to the observation that during this period of time SaOS-2 cells densely attach to the Ti—Ca-polyP discs and form an almost homogenous mono-cell layer. Amazing is the finding that the cells growing on the Ti—Ca-polyP discs show the phenotypic morphology of cell spreading, a clear sign for an active cell metabolism and cell migration.

“About” shall mean +/−10% of the value as indicated.

A further aspect of this invention concerns the finding that the inventive Ca-polyP coatings are able to stimulate the functional activity of bone forming cells, as demonstrated by the increased steady-state levels of transcripts encoding for the carbonic anhydrase IX (CA IX) and for the ALP in cells grown on the coated metal surfaces (compared to untreated titanium surfaces), as quantified by qRT-PCR.

The enzyme CA is involved in the initiation of bone formation (formation CaCO₃ deposits; Müller W E G, et al. Induction of carbonic anhydrase in SaOS-2 cells, exposed to bicarbonate and consequences for calcium phosphate crystal formation. Biomaterials 2013; 34:8671-8680; Wang X H, et al. Enzyme-based bio silica and biocalcite: biomaterials for the future in regenerative medicine. Trends Biotechnol 2014; 32:441-447).

The ALP is an established marker for functionally active, mineral deposit forming osteoblasts (see: Wang X H, et al. Bio-silica and bio-polyphosphate: applications in biomedicine (bone formation). Curr Opin Biotechnol 2012; 23:570-578).

The data underlying this invention show that titanium oxidized Ti-6Al-4V scaffolds are inert matrices for bone-like SaOS-2 cells in vitro. This metal acquires bio-functional properties if coated with the morphogenetically active Ca-polyP polymer. The progress in the biological functionalization of this implant material with polyP offers not only the fabrication of individualized implants but also provides the advantageous property to match the mechanical properties of the hard and brittle metal implant with those of the softer bone and its surrounding tissue.

The chain length of the polyP can be in the range of about 3 to about 1000 phosphate units, preferably in the range of about 20 to about 200 phosphate units, and most preferred about 40 phosphate units.

The preferred composition of the Ca-polyP microparticles used in the inventive method is a stoichiometric ratio of 0.1 to 1 and 50 to 1 (phosphate to calcium), preferably of 1 to 1 and 10 to 1, and most preferred 7 to 1.

It was unexpected that the Ca-polyP microparticles are biologically active although their diameter (0.2 and 3 μm) is outside the range allowing receptor-mediated endocytosis (around 50 nm).

The polyP material is biodegradable and displays superior morphogenetic activity, compared to the Ca-polyP salts prepared by conventional techniques.

A further aspect of the inventive method is the application/use of this method for the fabrication of biologically active titanium alloy implants. Another aspect of the inventive method is the application/use of this method for the preparation of implants that stimulate osteoblast cell activity. Another aspect of the inventive method is the combined application/use of Ca-polyP coated titanium alloy surfaces and implants with gallium salts in order to exploit their enhancing, synergistic effect on the coatings prepared by application of the inventive method. Unexpectedly the inventor found that gallium salts such as gallium nitrate enhance the stimulatory effect of the biologically active Ca-polyP Ti-alloy coatings on the expression, steady-state levels of transcripts that characteristic for functionally active osteoblasts. This finding was surprising because it has been reported that gallium salts only modulate bone resorption by osteoclasts but do not affect or only marginally affect gene expression and ALP activity of osteoblasts (Verron E, et al. Gallium modulates osteoclastic bone resorption in vitro without affecting osteoblasts. Br J Pharmacol 2010; 159: 1681-1692).

A further aspect of this invention concerns the surprising finding that an amorphous polyP-containing material with superior properties compared to crystalline HA and achieving nearly the same biological activity (morphogenetic activity; stimulation of bone-related gene expression) like the polyP microparticles disclosed in GB1420363.2, can also be prepared if polyP is present at a certain concentration in a procedure that normally results in the synthesis of crystalline HA.

The method according to this invention developed by the inventor for the preparation of biologically active amorphous polyP-substituted CaP particles (aCaP-polyP) comprises the following steps.

a) Addition of an aqueous solution of a polyP salt to an aqueous solution of a phosphate source;

b) Addition of the resulting solution to a dissolved calcium salt;

c) Adjustment of the pH to an alkaline value, preferably about 10; and

d) Collection, washing and drying of the resulting precipitate formed after several hours, preferably at room temperature after 24 h.

The polyP salt is preferably sodium polyP (Na-polyP). The inventive polyP-substituted CaP particles (aCaP-polyP) are formed, if the amount of the polyP salt is higher than 5 wt. % of polyP salt, referred to the CaP preparation. As an example, optimal results have been achieved with polyP-substituted CaP particles (aCaP-polyP) with 10 wt. % of polyP salt.

The calcium salt and the phosphate source forming the CaP component of the inventive polyP-substituted CaP particles (aCaP-polyP), prepared according to the inventive method, can be calcium chloride (CaCl₂) and ammonium phosphate dibasic [(NH₄)₂HPO₄)], respectively.

Optimal results were achieved with polyP-substituted CaP particles (aCaP-polyP) prepared by the inventive method, wherein the amount of the calcium salt and the amount of the reagent serving as phosphate source is calculated in order to obtain the Ca/P molar ratio for the CaP of 10:6.

The average size of the polyP-substituted CaP particles (aCaP-polyP) can be in the range of about 20 to about 300 nm, preferably in the range of a size of about 70 to about 120 nm.

A further aspect of this invention concerns the finding that the inventive polyP-substituted CaP particles (aCaP-polyP) are able to stimulate the functional activity of bone forming cells, as demonstrated by the increased steady-state levels of transcripts encoding for the collagen type I (COL-I) and for the alkaline phosphatase (ALP) in bone forming SaOS-2 cells, as quantified by qRT-PCR.

Unexpectedly, these polyP-substituted CaP particles (aCaP-polyP) are biologically active although their diameter (70-120 nm) is higher than the diameter of particles that can be taken up by receptor-mediated endocytosis (approximately 50 nm).

The polyP-substituted CaP particles (aCaP-polyP) are biodegradable and display superior morphogenetic activity, compared to the HAcrystals prepared by conventional techniques.

A further aspect of the inventive method is the application of this method for the fabrication of biologically active implant materials. Another aspect of the inventive method is the application of this method for preparation of artificial bone implants that stimulate osteoblast cell activity.

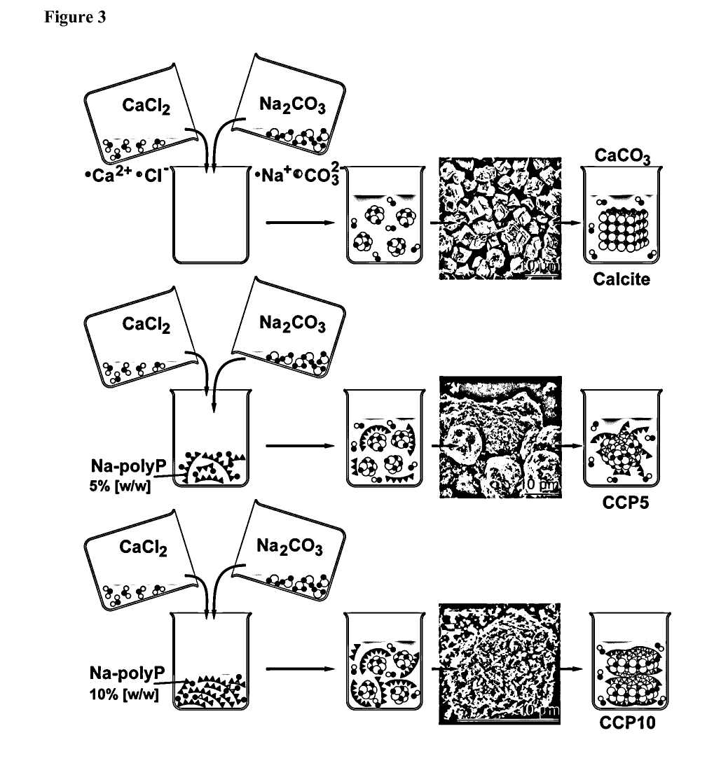

Another aspect of the invention concerns the production of an ACC polymorph that contains a small amount of vaterite. The inventor added the Na⁺ salt of the anionic polymer polyP to the precursors of CaCO₃ (CaCl₂ and Na₂CO₃) during the synthesis of ACC (FIG. 3). Surprisingly, the inventors found that polyP prevented, at a final concentration of 10%, the transformation process of ACC to its crystalline polymorphs vaterite, aragonite and calcite almost totally.

Both the CaCO₃ solids and the polyP physiological metabolite, tested separately, have osteogenic potential and could serve as constituents of bioactive bone grafts. In turn, the scaffold developed exploits not only the morphogenetic potential of polyP but also utilizes the property of this polymer to freeze the CaCO₃ solids at the ACC stage. This material is superior to calcite with respect to the osteogenic activity; it strongly induces the expression of the gene encoding for ALP (marker for bone formation) via stimulation of osteoblasts. This result has been obtained from studies with bone-like SaOS-2 cells and also with MSC.

Moreover, the inventor demonstrated that ACC/polyP strongly upregulates the expression of BMP2 (inducer of bone formation) by osteoblasts. Even more important: They surprisingly found that ACC increases the induction of BMP2 expression by polyP in a “synergistic” way, resulting in a faster rise of the BMP2 transcript levels. It can be expected that this effect of the inventive ACC/polyP microparticles will result in a faster healing of bone defects.

The ACC/polyP material is not only biocompatible but also supports the cellular regeneration of the impaired implant region. To assess the bio compatibility of the ACC/polyP material in vivo, the inventor encapsulated the inventive material into PLGA microspheres. In parallel, control spheres remained without ACC/polyP. The pearls/microspheres were inserted in the muscles of the back of rats. After an observation period of 2, 4, and 8 weeks tissue samples were taken from the rats and inspected microscopically after slicing and staining with Mayer's hematoxylin. The inspections show that in the animals with the microspheres containing the ACC/polyP material, an advanced repopulation of the implant region with cells became evident after 4 weeks and 8 weeks, resp. In contrast, the microspheres lacking ACC/polyP were devoid of any cells. These results were supported by measurements of the hardness (median RedYM stiffness) of tissue samples of the implant region, which revealed a significant increase by 1.8-fold compared to control after a period of 8 weeks (81% of the value measured in muscle samples before implantation).

The preferred method for the preparation of the inventive ACC/polyP material developed by the inventor comprises the following steps.

a) Preparation of a solution containing a polyP salt in, for example, one liter of 0.1 M NaOH

b) Addition of 0.5 mol/L of Na₂CO₃ to this solution

c) Dilution of the resulting solution with the 1.5 volume of deionized water

d) Rapid mixing of this solution with the same volume of an aqueous solution containing 0.5 mol/L of CaCl₂.2H₂O (resulting in an equimolar concentration ratio between calcium ions and carbonate ions); and

e) Filtration and drying of the precipitate after washing with acetone at room temperature

The preferred concentration of the polyP salt in the 0.1 M NaOH solution used for the preparation of the inventive ACC/polyP microparticles is in the range of 0.001 mol/L to 1.0 mol/L, preferably in the range of 0.01 mol/L to 0.1 mol/L (based on phosphate units).

Optimal results were achieved, if the concentration of the polyP salt in the 0.1 M NaOH solution used for the preparation of the inventive ACC/polyP microparticles is 0.025 mol/L or, even better, 0.05 mol/L (based on phosphate). The resulting preparations are termed “CCP5” and“CCP10”, respectively. The polyP salt is preferably Na-polyP.

The chain length of the polyP can be in the range of 3 to about 1000 phosphate units. Optimal results are achieved with polyP molecules with an average chain length of approximately 10 to about 100 phosphate units, and within this range optimally about 40 phosphate units.

A further aspect of this invention concerns the finding that the inventive ACC/polyP particles exhibit osteogenic activity by inducing the expression of the genes encoding for ALP and for BMP2 in bone-forming SaOS-2 cells, as quantified by qRT-PCR.

The ACC/polyP particles are biodegradable and display superior morphogenetic activity, compared to calcite which is rapidly formed from ACC in the absence of polyP.

The inventor demonstrated that, using an ACC formulation with 10% [w/w] polyP (“CCP10”), the release of Ca²⁺, and simultaneously of CO₃², is fast during the first 48 h of incubation, allowing the release of the biologically active anions CO₃² and PO₄³ from the scaffold. The ortho-phosphate will be enzymatically liberated from polyP, as previously demonstrated by the inventor (Müller W E G, Wang X H, Diehl-Seifert B, Kropf K, Schloßmacher U, Lieberwirth I, Glasser G, Wiens M, Schröder H C (2011) Inorganic polymeric phosphate/polyphosphate as an inducer of alkaline phosphatase and a modulator of intracellular Ca²⁺ level in osteoblasts (SaOS-2 cells) in vitro. Acta Biomater 7:2661-2671). In turn, the CO₃²⁻ as well as the HCO₃⁻ anions induce the mineralization process onto bone-forming cells, very likely via modulating the efficiency of the HCO₃⁻/Cl⁻ anion exchanger, inserted into the plasma membrane not only of osteoclasts but also of osteoblasts.

A further aspect of the inventive method is the application of this method for the fabrication of biologically active implant materials. Another aspect of the inventive method is the application of this method for preparation of artificial bone implants that stimulate osteoblast cell activity. Furthermore, another aspect of the invention described herein is an implant prepared by application of the inventive method.

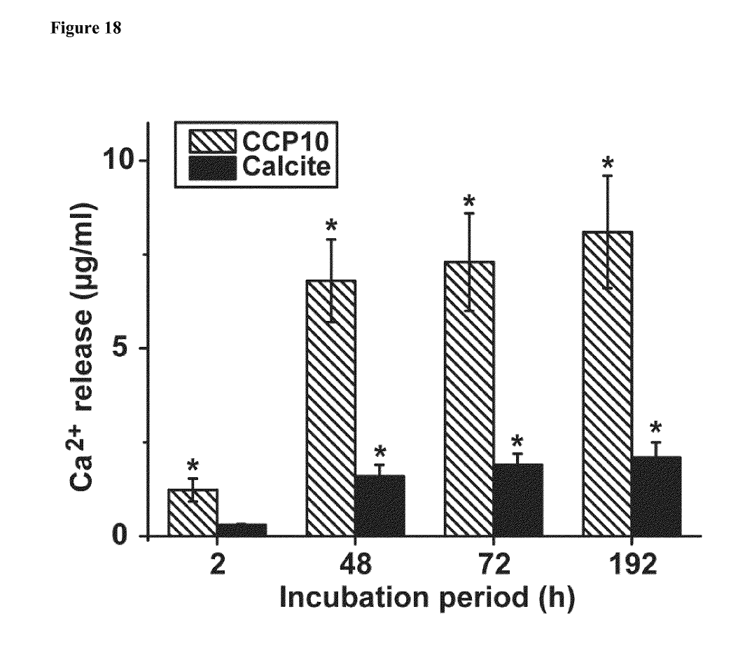

The inventive method to stabilize the metastable ACC with polyP also allows the application of ACC/polyP particles as a dietary supplement. As demonstrated by the inventor, e.g. see FIG. 18, these particles, e.g. “CC10” release calcium over prolonged incubation periods, in contrast to the crystalline calcite polymorph.

Therefore, the ACC/polyP particles according to this invention can also be used as a dietary supplement for treatment of calcium deficiency.

Accordingly, another aspect of this invention is the use of the stabilized ACC (ACC/polyP) as a dietary supplement for prophylaxis/therapy of osteoporosis.

Calcium plays an important role in many biological processes, for example in intracellular signalling, muscle contraction, neuronal transmission, and vasoconstriction/vasodilatation. ACC stabilized by polyP can serve as an easily available food supplement for calcium for prophylaxis/therapy of many pathological conditions, associated with disturbances of calcium metabolism.

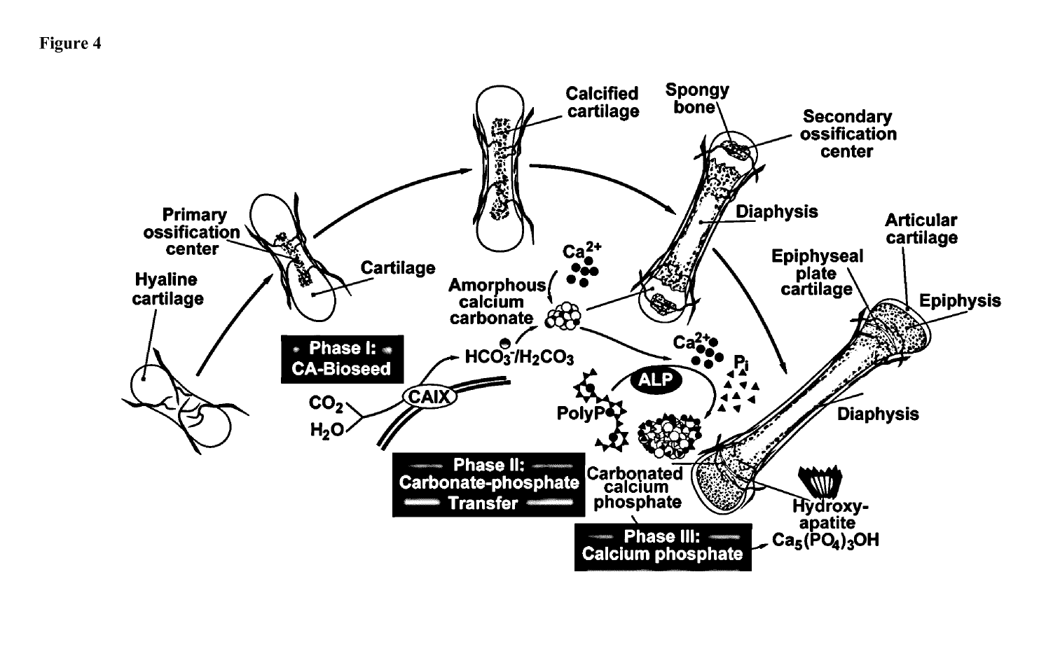

Based on recent findings on the CaCO₃ nature of the bio seeds, the anion exchange of CO₃²⁻ by PO₄³⁻ and the supply of ortho-phosphate from polyP the following series of mechanistically distinct processes can be described during bone formation (FIG. 4). In the first phase during bone mineral deposition, like in the endochondral ossification, the cartilage in the metaphysis comprising the growth center between the epiphysis and the diaphysis of the long bone, calcifies. It is likely that this process of calcification is enzymatically driven by CA-II and/or CA-IX. Secondly, platelets that accumulate besides of the osteoblasts both in regions of bone formation and also at bone fracture sites release polyP into the extracellular space where the polymer undergoes ALP-mediated exohydrolysis under the release of ortho-phosphate. Thirdly, the available phosphate units, formed in a spatial vicinity to the bioseed synthesis, serve as the source for the formation of ACP. As sketched in this scheme the inventive material is a promising biocompatible and osteogenic scaffold that provides both the substrate for the bioseed development (CaCO₃[CO₃²⁻]) and for the subsequent transformation to the calcium phosphate (polyP [PO₄³⁻]).

Thus, in summary, the present invention relates to a method for the production of biologically active coatings of titanium alloys, comprising the following steps: a) Preparing Ca-polyP microparticles by mixing of an aqueous solution of Na-polyP with an aqueous solution of calcium chloride dihydrate (CaCl₂.2H₂O) for several, preferably 3, hours at elevated temperature, preferably at 90° C., under formation of a colloidal suspension; b) Coupling said Ca-polyP microparticle colloidal suspension to a suitable titanium alloy scaffold using a silane coupling agent; and c) adjusting of the pH value of the suspension of b) to a slightly alkaline value, preferably 8.0, to allow binding of polyP to the silane-functionalized metal scaffold via Ca²⁺ ionic bond formation. The titanium alloy can be Ti-6Al-4V. The silane coupling agent can be (3-aminopropyl)trimethoxysilane [APTMS].

The present invention also relates to a method for the preparation of biologically active amorphous polyphosphate-substituted calcium phosphate particles (“aCaP-polyP”) comprising the following steps: a) Adding of an aqueous solution of a polyphosphate salt to an aqueous solution of a phosphate source; b) Adding of the resulting solution to a dissolved calcium salt; c) Adjusting the pH to an alkaline value, preferably 10; and d) Collecting, washing, and drying of the resulting precipitate formed after several hours, preferably at room temperature after 24 h. The polyphosphate salt can be sodium polyphosphate.

The present invention also relates to a method for the preparation of biologically active amorphous calcium carbonate (ACC)-polyphosphate microparticles, comprising the following steps: a) Preparing of an aqueous solution of a polyphosphate salt in about 0.1 M sodium hydroxide; b) Adding of about 0.5 mol/L of sodium carbonate to said solution; c) Diluting of the resulting solution with about 1.5 volumes of deionized water; d) Mixing of said solution with the same volume of an aqueous solution containing calcium chloride, so that an about equimolar concentration ratio between calcium ions and carbonate ions results; e) Washing with a lower alkyl ketone, such as acetone, at about room temperature; and f) Filtering and drying of a precipitate as formed. the concentration of the polyphosphate salt in step a) is in the range of about 0.001 mol/L to about 1.0 mol/L, preferably in the range of about 0.01 mol/L to about 0.1 mol/L, based on phosphate. Preferably, the concentration of the polyphosphate salt in step a) is about 0.025 mol/L or about 0.05 mol/L, based on phosphate.

Preferred are methods wherein the chain length of the polyphosphate is in the range of about 3 to about 1000 phosphate units, preferably in the range of about 10 to about 100 phosphate units, and most preferred about 40 phosphate units. Preferred are methods wherein the amount of the polyphosphate salt is higher than 5 wt. %, preferably 10 wt. %, referred to the calcium phosphate preparation.

Preferred are methods wherein the calcium salt is calcium chloride (CaCl₂) and the phosphate source is ammonium phosphate dibasic [(NH₄)₂HPO₄)].

In the invention, the calcium polyphosphate microparticles can be characterized by a stoichiometric ratio between 0.1 to 1 and 50 to 1 of phosphate to calcium, preferably of between 1 to 1 and 10 to 1, or by a stoichiometric ratio of 7 to 1 of phosphate to calcium.

Preferred are methods wherein the amount of the calcium salt and the amount of the reagent serving as phosphate source is calculated in order to obtain the Ca/P molar ratio for the calcium phosphate of 10:6. Preferred are methods wherein the average size of the calcium polyphosphate microparticles is in the range of about 0.1 to about 30 μm, preferably between 0.8 and 3 μm. Preferred are methods wherein the average size of the polyphosphate-substituted calcium phosphate particles (“aCaP-polyP”) is in the range of about 20 to about 300 nm, preferably about 70 to about 120 nm.

Preferred are methods that further comprise the step of producing biologically active titanium alloy implants. Also preferred are methods that further comprise the step of producing a biologically active implant material. Also preferred are methods that further comprise the step of including at least one gallium salt into said implant. Preferably, said biologically active implant material is an artificial bone implant. Preferably, said biologically active implant material is an artificial bone implant.

The present invention also relates to an implant prepared by the method according to the invention, or a. stabilized ACC composition produced by the method according to the invention.

Preferably, the coating as produced according to invention can be used as an implant, optionally in combination with at least one gallium salt, or as a food or dietary supplement (e.g. ACC composition). The stabilized ACC composition is for use in the treatment of calcium deficiency, or for use in the prophylaxis and/or therapy of osteoporosis.

The invention will now be described further in the following preferred examples, nevertheless, without being limited thereto. For the purposes of the present invention, all references as cited herein are incorporated by reference in their entireties.

FIG. 1 shows a schematic outline of the formation of amorphous CaP (aCaP) from the precursors Ca²⁺, PO₄³⁻ and OH⁻. The aCaP undergoes maturation to crystalline HA, or in the presence of <10 wt. % polyP likewise to crystalline CaP (see insert at bottom, showing CaP crystals; SEM image). If the content of polyP increases to ≥10 wt. % polyP in the CaP precipitates spheroidal amorphous aCAP-polyP is formed (see insert at top; SEM image).

FIG. 2 shows a scheme of the binding of polyP to titanium discs using the silane coupling agent APTMS. The titanium alloy Ti-6Al-4V is etched with HCl and the hydroxyl groups, exposed onto the titanium discs, are cross-linked with the silane coupling agent APTMS that forms Ca²⁺-bridges to polyP. After dehydration/polycondensation the coupling agent still contains a free, reactive amine group that might be used for further coupling to active components, e.g. via 1-ethyl-3-(3-dimethylaminopropyl)carbodiimide. During this process the metal surface is covalently linked with the silane that in turn allows binding of polyP via Ca²⁺ ionic bridges.

FIG. 3 shows a scheme of the preparation of calcite and CaCO₃ supplemented with polyP. The inserts show the SEM images of the respective product.

FIG. 4 shows a scheme of the process of endochondral ossification and the proposed phases of bone mineral deposition. After penetration of blood vessels the hyaline cartilage at the primary ossification centers in the diaphysis starts to calcify. The formation of spongy bone at the secondary ossification centers in the epiphyses starts later. Two regions of hyaline cartilage remain, the articular cartilage at the surface of the epiphysis and the epiphyseal plate (growth region) between the epiphysis and diaphysis. The mineral deposition in the growth region is subdivided into phase I: Amorphous calcium carbonate (ACC) bioseeds are formed mediated by the membrane-associated CA-IX; phase II: PolyP released from platelets undergoes ALP-mediated hydrolysis under formation of ortho-phosphate for the carbonate-phosphate transfer reaction; and phase III: The phosphate units are used for the (carbonated) calcium phosphate formation.

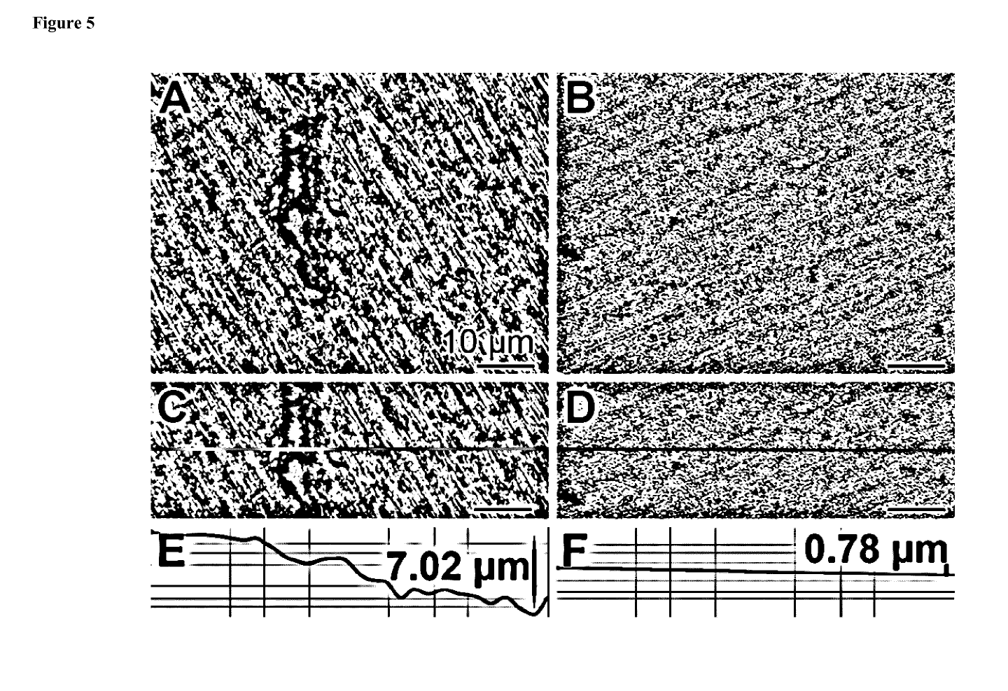

FIG. 5 shows the surface roughness of the titanium alloy discs (A, C, E) in comparison with the Ti—Ca-polyP discs (B, D, F). The surfaces of the discs were visualized by light microscopy and analyzed for roughness using the VK-analyser software. The tracks of the line-scans (C, D) are shown. The height profiles of representative regions are shown in E, F; the numbers indicate the maximal dimensions for the deviations within a normal vector straight line.

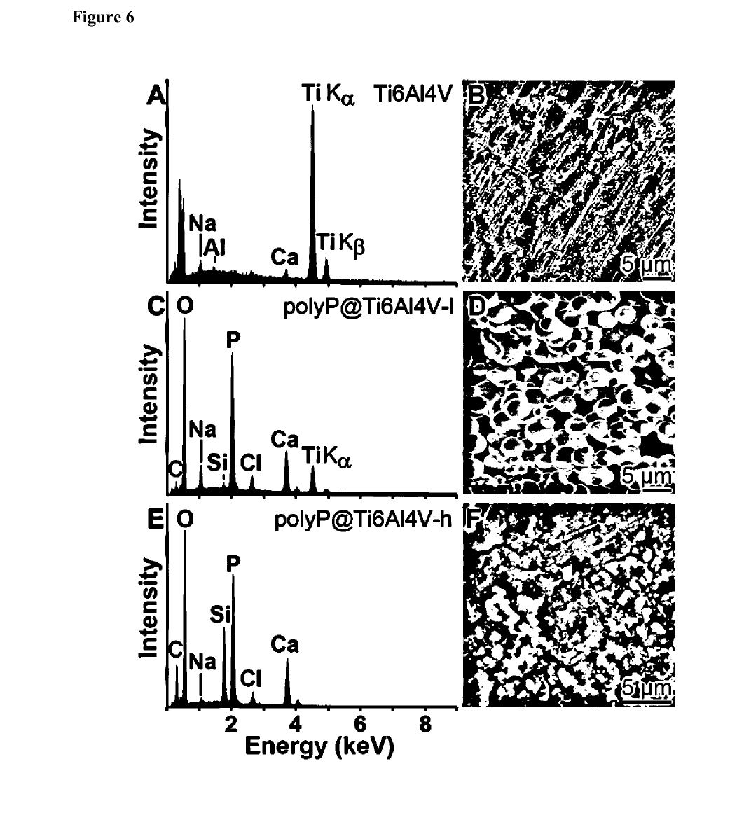

FIG. 6 shows the analysis of the element composition of the titanium and Ti—Ca-polyP discs by EDX spectroscopy (A, C, E) and SEM (B, D, F). (A, B) Untreated discs (Ti6Al4V); (C, D) Ti—Ca-polyP discs fabricated with the lower concentration of APTMS (1 mg/assay; polyP@Ti6Al4V-1) in the polyP and CaCl₂ reaction assay; and (E, F) Ti—Ca-polyP discs which have been coated in the presence of higher APTMS concentration (2 mg/assay; polyP@Ti6Al4V-h).

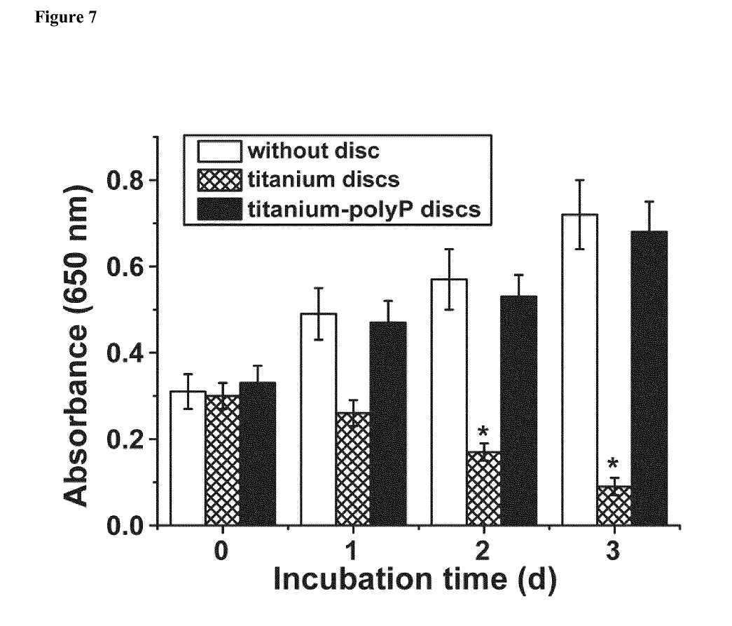

FIG. 7 shows the effect of titanium discs on growth of SaOS-2 cells. The cells were seeded, under otherwise identical conditions, into 24-well plates that did not contain titanium discs (open bars), titanium alloy discs (cross-striped bars) or Ti—Ca-polyP discs (filled bars). After an incubation period of 1, 2 and 3 d the cells were harvested and the viability of the cells was determined by the XTT assay. Data represent means±SD of ten independent experiments (* P<0.01).

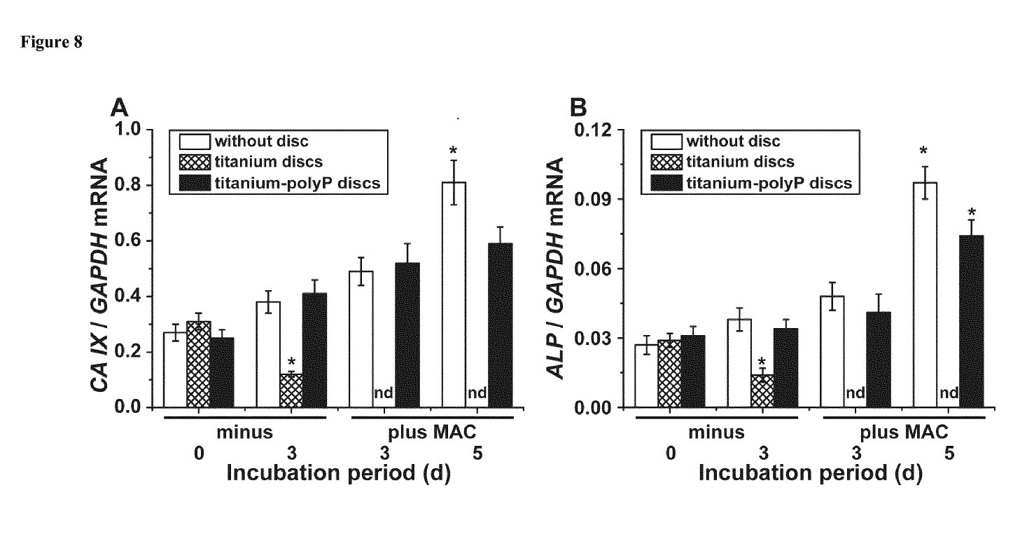

FIG. 8 shows the expression of the genes encoding for (A) CA IX and for (B) ALP. The expression values were normalized to the expression of GAPDH. The cells were cultivated either without any titanium discs (open bars), or either onto titanium alloy discs (cross-striped bars) or on Ti—Ca-polyP discs (filled bars). The cultures were incubated at first in the absence of the MAC for 3 d; then they were transferred to medium, supplemented with the MAC, and the incubation was continue for additional 3 or 5 d, as outlined. Then the cells were harvested, RNA was extracted and subjected to qRT-PCR for determination of both CA IX and ALP transcripts; the expression of GAPDH served as reference. Data are expressed as mean values±SD for five independent experiments; each experiment was carried out in duplicate (* P<0.01). nd, not detectable.

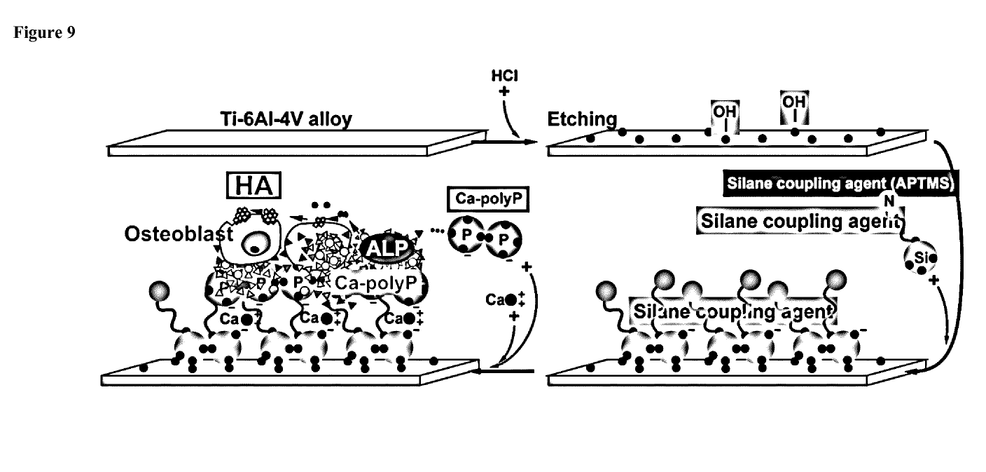

FIG. 9 shows the coating of titanium discs with morphogenetically active Ca-polyP. The metal material (Ti-6Al-4V) acquire bio-functional properties if coated with the morphogenetically active Ca-polyP polymer. During the process the titanium surfaces becomes etched, resulting in the exposure of hydroxyl groups. At a pH of 8 they form covalently linkages with siliane coupling agents, e.g. APTMS. Under this environment Ca²⁺-ionic bridges are formed between the silane and polyP. Those coated titanium discs allow bone-like SaOS-2 cells to settle on and induce them to gene expression (CA IX and ALP); these enzymes are crucially involved in bone-mineral/hydroxyapatite (HA) deposition.

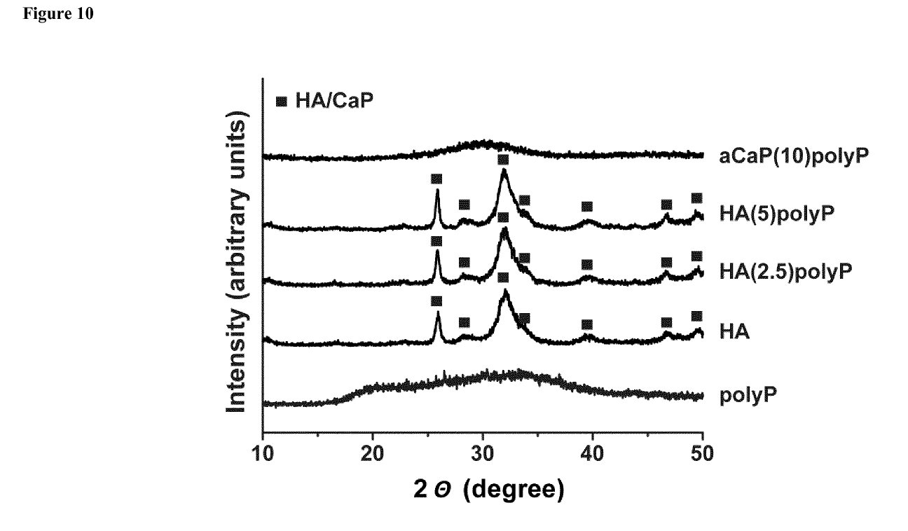

FIG. 10 shows the diffraction patterns taken from pure Na-polyP “polyP” and pure “HA”, as well as from HA, prepared in the presence of different amounts of Na-polyP, 2.5 wt. % as in “HA(2.5)polyP”, 5 wt. % in “HA(5)polyP”, and 10 wt. % in “aCaP(10)polyP”. The respective patterns are given from the bottom to the top. No diffraction signals are seen for “polyP” and “aCaP(10)polyP”. The diffraction peaks characteristic for HA or crystalline CaP are highlighted (▪).

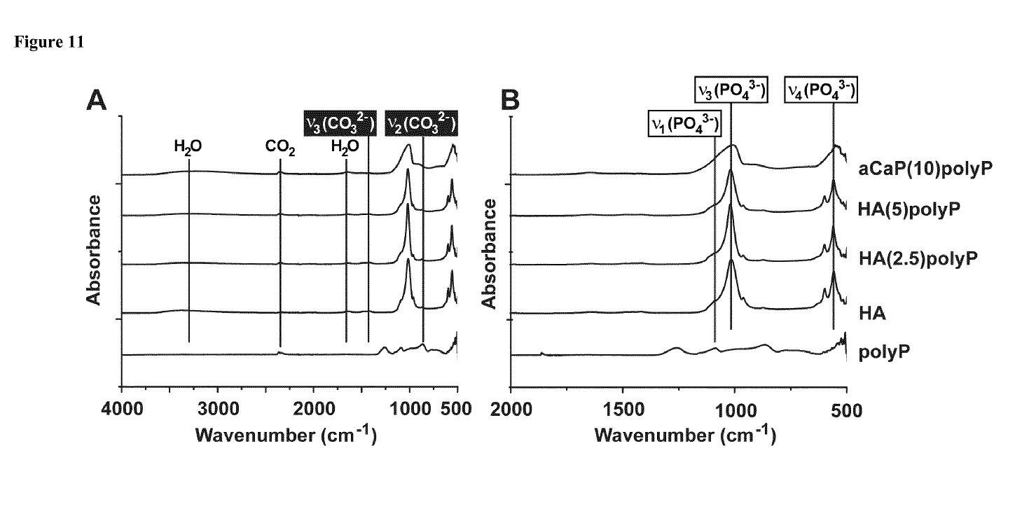

FIG. 11 shows the FTIR spectra for “polyP” and “HA”, as well as for CaP samples, in which ortho-phosphate has been partially substituted by polyP, “HA(2.5)polyP”, “HA(5)polyP” and “aCaP(10)polyP”. Some vibration bands for CO₃²⁻ and PO₄³⁻ are marked; in addition, the regions for the H₂O and CO₂ bands are indicated. (A) Wavenumber range (in cm⁻¹) between 4000 and 500; (B) enlargement of the segment 2000 to 500 cm⁻¹.

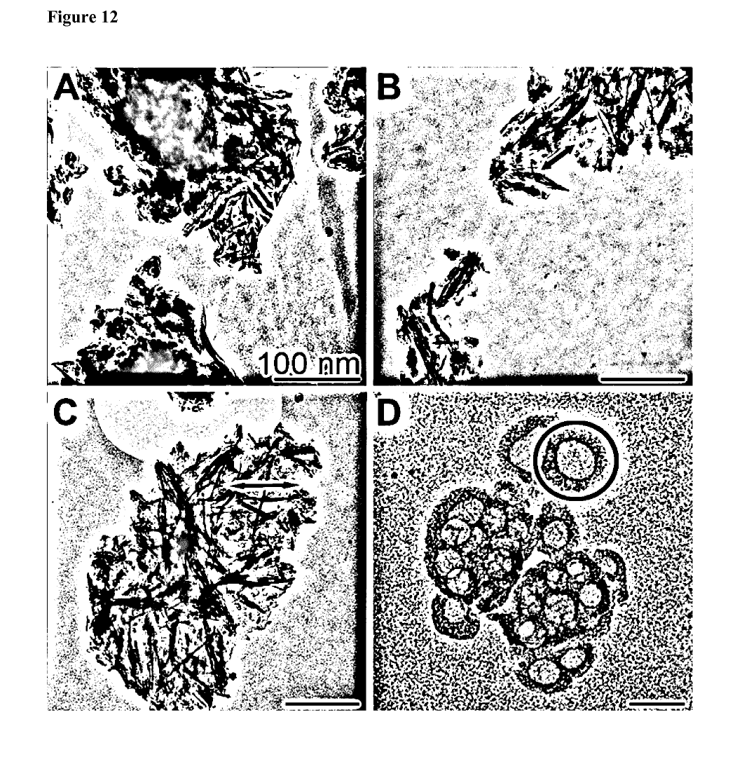

FIG. 12 shows the TEM micrographs of the polyP and CaP particles. (A) “HA” crystals; (B and C) “HA(2.5)polyP” and “HA(5)polyP” crystals; and (D) “aCaP(10)polyP” amorphous spheroidal particles.

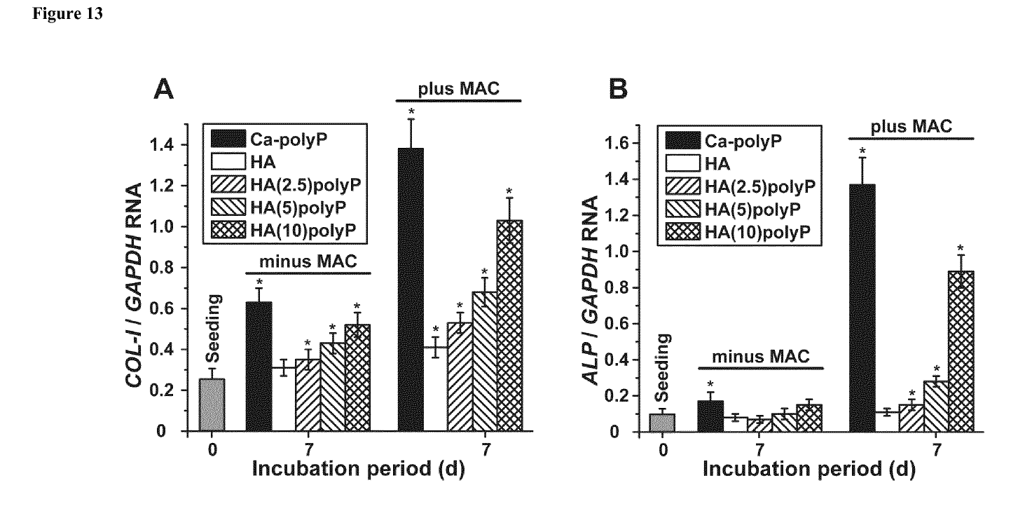

FIG. 13 shows the steady-state expression levels of the genes, encoding (A) for collagen type I (COL-I) or (B) for alkaline phosphatase (ALP) in SaOS-2 cells. The cells are exposed to 10 μg/1 mL polyP nanoparticles “aCa-polyP-NP” (filled bars), or to 100 mg/mL of “HA” (open bars), “HA(2.5)polyP” (right hatched bars), “HA(5)polyP” (left hatched bars) or “aCaP(10)polyP” (cross-hatched bars). After the initial incubation for 3 d in the standard medium/serum, the cells were transferred to culture medium/serum lacking (minus MAC) or containing MAC (plus MAC). After the 7 d incubation period the cells were harvested, the RNA extracted and subsequently used for qRT-PCR analyses. The expression values are given as ratios to the reference gene GAPDH. The results are means from 5 parallel experiments; * P<0.01.

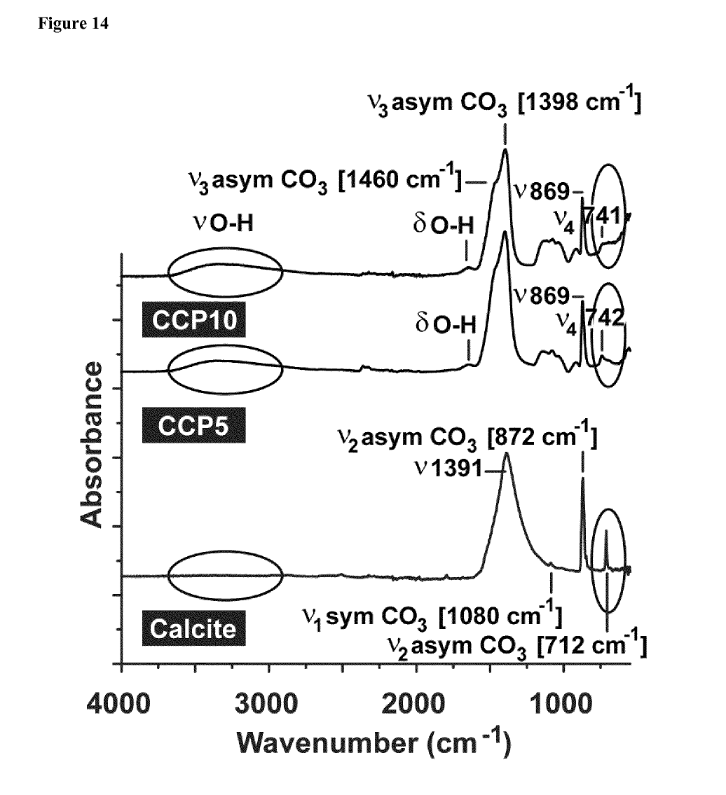

FIG. 14 shows the FTIR spectra of calcite as well as “CCP5” (0.05 g of Na-polyP/assay) and “CCP10” (0.1 g of Na-polyP). The major distinguishing vibration regions/signals for calcite versus ACC, the vibration range for O—H (around 3250 cm⁻¹) and the asymmetric ν₂ line for CO₃ at 725 cm⁻¹ are circled.

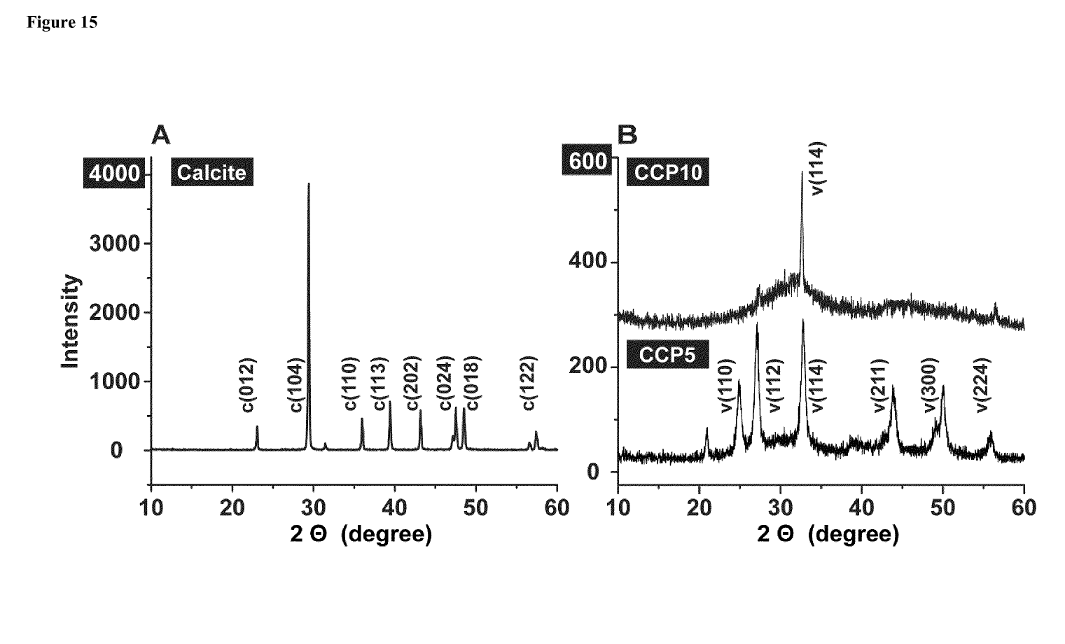

FIG. 15 shows the XRD pattern obtained from (A) calcite and (B) the two CaCO₃ preparations, containing two different concentrations of polyP, “CCP5” or “CCP10”. The characteristic signals are highlighted and marked with the respective Miller indices, given in parentheses. Please note the different scale of the ordinate captions between (A) and (B).

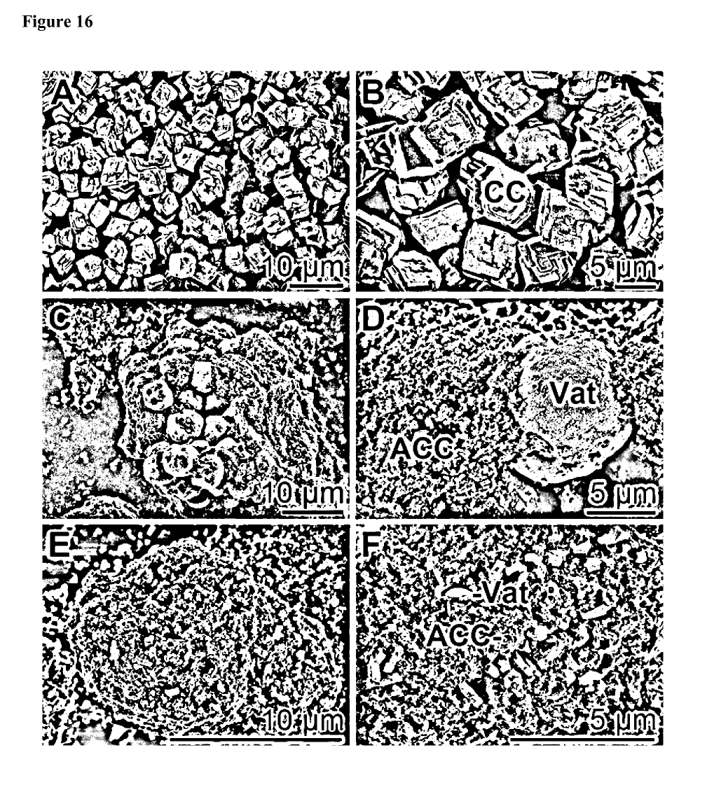

FIG. 16 shows the morphology of the solids formed from CaCl₂.2H₂O and Na₂CO₃; SEM analysis. (A and B) In the absence of polyP calcite crystals are formed. This morphology is changed after addition of polyP during the precipitation process. (C and D) In the presence of 5% polyP, the “CCP5” particles show a spherical appearance. (E and F) At 10% polyP, “CCP10”, the solids show a platelet-like shape, which corresponds to vaterite crystals (Vat).

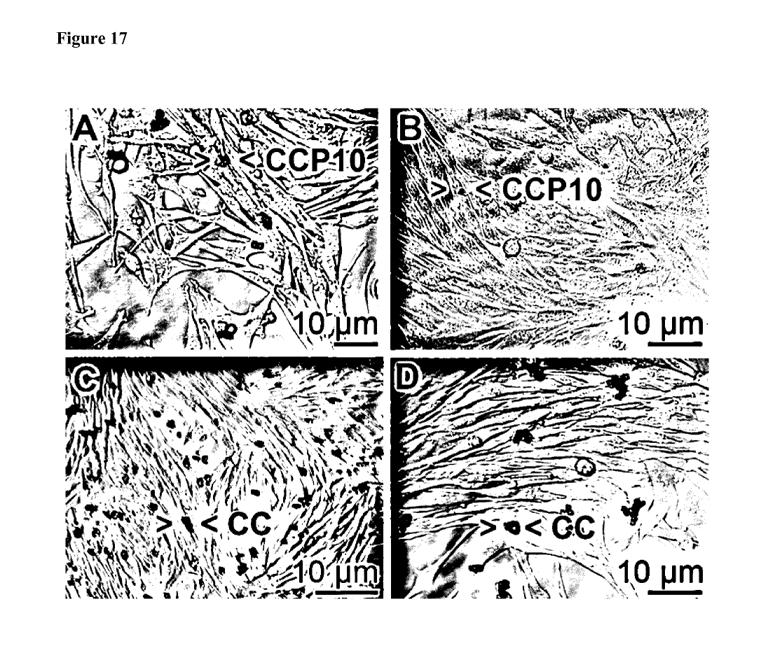

FIG. 17 shows the growth pattern of SaOS-2 cells in the presence of 50 μg/mL of “CCP10” (A and B) or calcite (C and D) after a 3 d incubation period. The cells were identified by phase contrast/Nomarski optics. The CaCO₃ particles in the assays became visible in the phase contrast images and are marked (> <).

FIG. 18 shows the release of Ca²⁺ from the CaCO₃ particles. “CCP10” or calcite was incubated in Tris-HCl buffer (pH 7.4) for various time periods and the supernatant was analyzed for Ca²⁺ concentration. The results are means from 6 parallel experiments; * P<0.01.

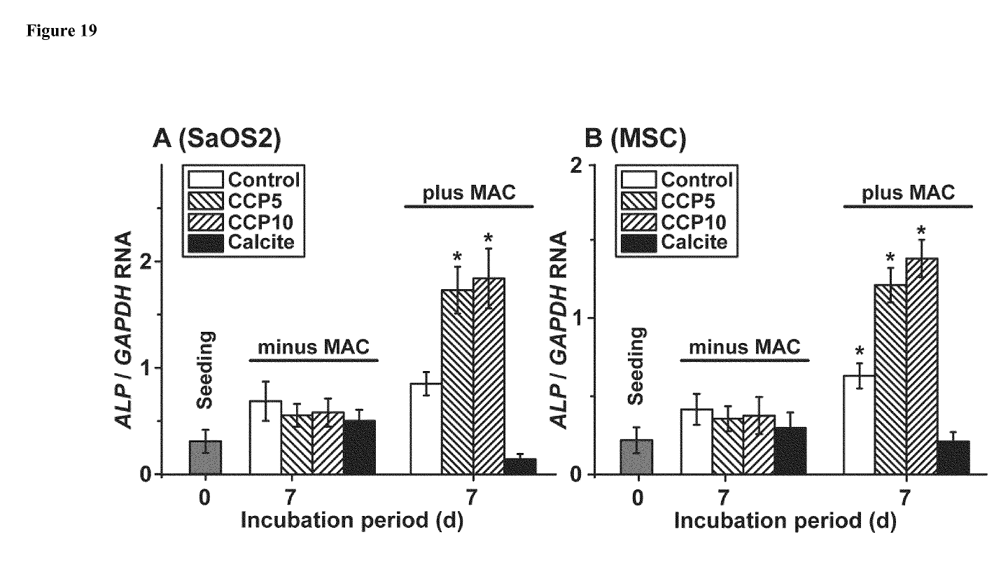

FIG. 19 shows the steady-state expression levels of the ALP gene both in (A) SaOS-2 cells and in (B) MSCs. The cells remained without any CaCO₃ solids (control), or were exposed to 50 μg/mL of “CCP5” (left hatched bars), “CCP10” (right hatched bars), or calcite (filled bars). After the 3 d pre-incubation period in the absence of MAC the cells were continued to be incubated in the absence of MAC (minus MAC) or were exposed to MAC (plus MAC). After the 7 d incubation the cells were harvested, their RNA extracted and subjected to qRT-PCR analyses. The expression values are given as ratios to the reference gene GAPDH. The results are means from 5 parallel experiments; * P<0.01; the values are computed against the expression measured in cells during seeding.

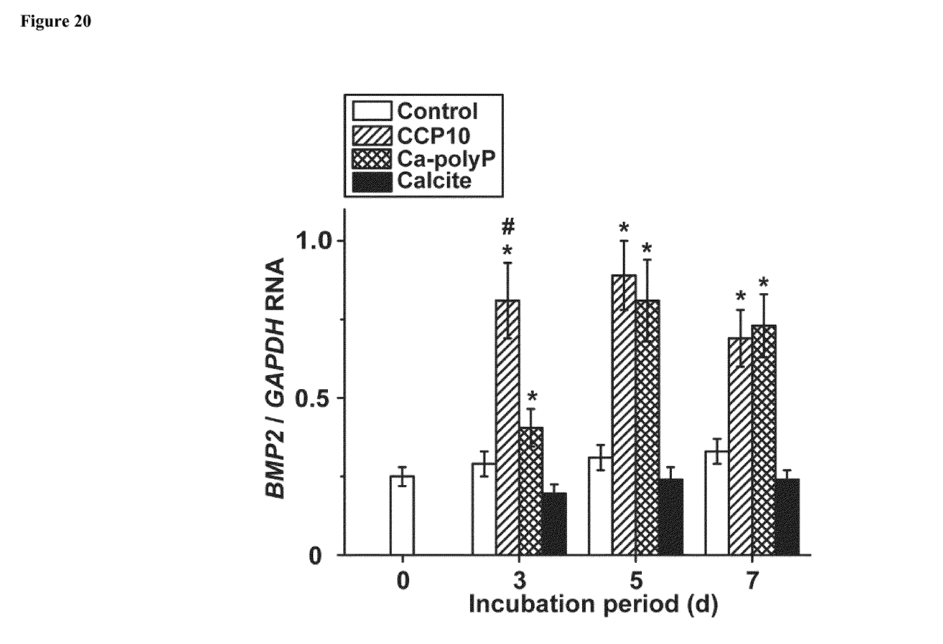

FIG. 20 shows the steady-state expression levels of the BMP2 gene both in SaOS-2 cells in the presence of “CCP10” and polyP (Ca²⁺ complex). The cells remained without any additive (control), or were exposed to 50 μg/ml of “CCP10” (right hatched bars), 5 μg/ml of polyP (Ca²⁺ complex; 50 μM phosphate units; cross hatched bars), or 50 μg/ml of calcite (filled bars). After the 3 d pre-incubation period in the absence of MAC the cells were continued to be incubated in the presence of MAC for up to 7 days, and the expression BMP2 was analyzed by qRT-PCR. The expression values are given as ratios to the reference gene GAPDH. The results are means from 5 parallel experiments; * P<0.01; the values are computed against the expression measured in cells during seeding (day 0); # P<0.01 (only for “CCP10”); the values are computed against the expression measured in cells with polyP (Ca²⁺ complex) at the respective incubation periods.

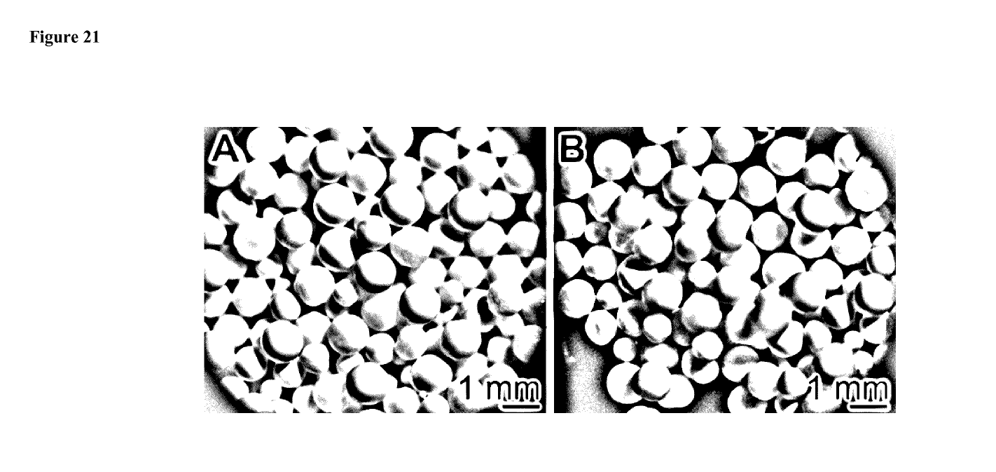

FIG. 21 shows the morphology of the microspheres; (A) control spheres “cont-mic” and (B) polyP loaded spheres, “polyP-mic”.

### EXAMPLES

In the following examples, the inventive method is described only for polyP molecules with a chain length of 40 phosphate units. Similar results can be obtained by using polyP molecules with lower and higher chain lengths, such as between about 20 to 100 units.

### Titanium-Ca-polyP (Ti—Ca-polyP) Discs

Titanium alloy (Ti-6Al-4V) disks were etched to allow cross-linking with the silane coupling agent APTMS (FIG. 2). In the second step the discs were covered with polyP via Ca²⁺ ionic bridges. Finally the specimens, the Ti—Ca-polyP discs, were dried at 100° C. We used—on purpose—the silane coupling agent APTMS to provide a further functional group, an amine group, to couple also bioactive peptides to the polyP-coated metal surface. The functionalization of the titanium discs has also been performed with 3-(trimethoxysilyl)propyl methacrylate successfully allowing a polyP-titanium coating only.

A comparison between the titanium alloy discs and the Ti—Ca-polyP discs (light microscopic images) revealed that, in contrast to the dark gray surface color of the titanium alloy discs, the Ti—Ca-polyP discs have a shiny silver-white appearance. After the coating of the surfaces of the discs with polyP they lose their high roughness. While the untreated discs have an average roughness of ≈5.5 μm with a maximum of 7.02 μm (FIG. 5A, C, E) the polyP coated discs expose a surface roughness of 0.78 μm in maximum (FIG. 5B, D, F).

Element-specific analyse of the surfaces of the titanium discs was performed by EDX spectroscopy (FIG. 6). The surface of the non-treated discs showed the dominant K_{α} peak for titanium at 4.5 keV and the lower K_{β} peak at 4.9 keV (FIG. 6A). The morphology of the surface is marked rough (FIG. 6B). If the titanium discs, after etching and reacting with the lower concentration of APTMS (1 mg/assay), are examined after an incubation in the coating solution with polyP and CaCl₂, Ca-polyP microparticles (Müller W E G, Tolba E, Schröder H C, Diehl-Seifert B and Wang X H. Retinol encapsulated into amorphous Ca²⁺ polyphosphate nanospheres acts synergistically in MC3T3-E1 cells. Eur J Pharm Biopharm 2015; 93:214-223) can be resolved by SEM (FIG. 6D). The size of the particles varies between 0.8 and 3 μm. After drying the discs at 100° C. the EDX determinations were performed. A representative spectrum (FIG. 6C) shows the now dominant K_{α} peak for phosphorus at 2.01 keV. In addition, the calcium peak (3.69 keV) is detectable. The titanium peak (4.5 keV) is recordable as well. If the disc samples coated with polyP after addition of the higher amount of APTMS (2 mg/assay) are inspected by SEM an almost homogeneous polyP surface can be visualized by SEM (FIG. 6F). This observation is supported by the EDX measurements that revealed a (almost) complete disappearance of the titanium peak (FIG. 6E), while the phosphorus and calcium peaks become dominant.

### Durability of the Ca-polyP Coat

The surface coat of the polyP was measured by the determination of Ca²⁺ release from the coated discs in SBF (lacking Ca²⁺ as component), as described under “Methods”. In parallel assays, the release of Ca²⁺ from Ti—Ca-polyP discs as well as from untreated titanium discs (control) was measured. As an additional control one Ti—Ca-polyP disc each was inserted in the SBF incubation medium supplemented with 1 μg/ml of ALP; all samples were incubated at 37° C. At time zero in all three assays the Ca²⁺ concentration was <3 μg/ml. After one d in the incubation medium the amount of Ca²⁺ was determined as follows: Ti—Ca-polyP discs: <3 μg/ml (<3 μg/ml [control]; 12±3 μg/ml [Ti—Ca-polyP discs+ALP]); 5 parallel assays were performed. The Ca²⁺ release increased slightly in assays containing the Ti—Ca-polyP discs after a 3 d incubation period, in contrast to the assays of Ti—Ca-polyP discs together with ALP. The following values are measured: 5.2±0.8 μg/ml [Ti—Ca-polyP discs] (<3 μg/ml [control]; 86.9±3.2 μg/ml [Ti—Ca-polyP discs+ALP]). After 12 d in the incubation assay the values are as follows: 9.7±1.2 μg/ml [Ti—Ca-polyP discs]; <3 μg/ml [control]; 153.1±17.1 μg/ml [Ti—Ca-polyP discs+ALP].

### Growth of SaOS-2 Cells on the Titanium Discs

The overall growth rate of the bone-like SaOS-2 cells was determined by the XTT assay as described under “Methods”. The cells were seeded at a density of 3×10⁴ cells/well (2 ml assays) for all three parallel series of experiments; assays without titanium discs, titanium alloy discs, Ti—Ca-polyP discs (FIG. 7). Already after a 1-d incubation period the density of the cells increased from 0.3 absorbance units to 0.49±0.6 units (assays without discs) and 0.47±0.05 units (with Ti—Ca-polyP discs), while the density in the assays with titanium alloy discs decreased to 0.26±0.03 units. This tendency increased during the following incubation days and reached values after 3-d incubation period of 0.09±0.02 (titanium alloy discs; significant reduction), 0.72±0.08 (absence of disc) and 0.68±0.07 (Ti—Ca-polyP discs). These data imply that the titanium surfaces are not supporting growth of the SaOS-2 cells, while the cells, growing on Ti—Ca-polyP discs showed the same growth kinetics like that of cultures without any discs.

The property of the discs, coated with polyP, being a very suitable matrix for SaOS-2 cells to grow onto, was also underscored by staining the surface of the discs. Titanium alloy discs, not coated with polyP were incubated for 3 d with SaOS-2 cells; after that period no cells could be visualized onto the discs. In contrast, if polyP-coated Ti—Ca-polyP discs are incubated in the presence of SaOS-2 cells for the same period of time an almost homogenous mono-cell layer is observed. A closer inspection at higher magnification revealed that the cells show the property of cell spreading, a characteristic sign for vital survival and growth of cells.

As a further support of the conclusion that SaOS-2 cells are growing readily onto Ti—Ca-polyP discs the areas, covered with cells, were analyzed for the distribution of elements carbon (C), titanium (Ti) and phosphorus (P). The semiquantitative determinations of the elements were performed by SEM-based EDX mappings. The localization of the cells was obtained by recording the back-scattered electrons. Within the regions where the cells grow a high accumulation for the element C is measured, while titanium and polyP are highlighted outside of the cell areas, at the surrounding surface of the discs onto which the cells grow.

### Expression of Carbonic Anhydrase IX and Alkaline Phosphatase

As a marker for the functional activity of the SaOS-2 cells, growing onto titanium discs, the expression of the two genes encoding for the enzymes carbonic anhydrase IX (CA IX) and alkaline phosphatase (ALP) was determined by quantitative qRT-PCR. The studies for the steady-state level of transcripts of CA IX in SaOS-2 cells growing for 3 d in the absence of the MAC showed for the cultures which contained titanium alloy discs a significant decrease of the expression levels from 0.31±0.03 (time at seeding) to 0.12±0.01, while the levels in the cells cultured in the absence of discs or the presence of the Ti—Ca-polyP discs increased from 0.27±0.02 and 0.25±0.03 to 0.38±0.04 and 0.40±0.05, respectively (FIG. 8A). A subsequent incubation of the cultures in the presence of the MAC resulted in an increase of the levels for the CA IX expression in assays that contained no discs or into which Ti—Ca-polyP discs have been submersed. After 5 d in the presence of the MAC a significant increase of the CA IX transcript level in cells in the absence of discs from 0.38±0.04 to 0.81±0.08 was detected. A pronounced increase of this gene was also found in cells, cultured onto Ti—Ca-polyP discs with 0.41±0.05 to 0.59±0.06. These results underline that the coating with polyP onto titanium implant material provides the materials with a biologically active surface (FIG. 9).

In parallel, the expression of the gene for the enzyme ALP was determined, likewise by qRT-PCR. Again the data (FIG. 8B) show that the expression level of ALP in culture containing the titanium alloy discs significantly decrease after a 3 d incubation period the absence of any discs. Later during the incubation the level is so low, that the expression cannot documented reliably. In contrast, in the presence of the MAC the steady-state expression of the ALP increases significantly, both in the assays without discs and in the assays with Ti—Ca-polyP discs; from 0.038±0.005 to 0.097±0.007 (at day 5 without discs) and from 0.034±0.004 to 0.074±0.007, respectively.

In a further set of experiments, the assays were performed in the presence of 100 μM gallium nitrate (see Table 1). The cells were cultivated either without any titanium discs, or either onto titanium alloy discs or on Ti—Ca-polyP discs, as described above, first in the absence of the MAC for 3 d and then in medium supplemented with the MAC for additional 5 d. The results revealed that in the absence of discs, the steady-state level of CA IX transcripts in SaOS-2 cells growing for 3 d in the absence of the MAC and subsequently for 5 d in the presence of the MAC increased from 0.24±0.05 to 0.89±0.11 (Table 1), compared to 0.27±0.02 to 0.81±0.08 in the absence of gallium nitrate (see also FIG. 8). A much stronger increase in the level of CA IX transcripts in the presence of gallium nitrate, compared to assays in the absence of the gallium nitrate was found, if the cells were cultured onto Ti—Ca-polyP discs; the steady-state level of CA IX transcripts increased from 0.27±0.04 to 0.96±0.15 (presence of 100 μM gallium nitrate), compared to 0.25±0.04 to 0.59±0.06 (absence of gallium nitrate; see also FIG. 8), indicating a strong synergistic effect of the Ca-polyP coating and the gallium salt (comparison of the assays without discs and with Ti—Ca-polyP discs).

Similar results were obtained, if the effect of gallium nitrate on the steady-state levels of ALP transcripts in the absence of discs and in the presence of titanium alloy discs or of Ti—Ca-polyP discs were determined. In the absence of the titanium discs, the addition of the gallium salt had only a small effect on the ALP transcript levels, compared to the assay without this additive, while in the presence of the Ti—Ca-polyP discs, again a strong synergistic effect on the Ca-polyP-caused increase of the ALP transcript levels was observed (increase from 0.027±0.003 to 0.115±0.007), if compared with the assay without this supplement (increase from 0.030±0.003 to 0.074±0.007; see also FIG. 8). No detectable or only very small transcript levels were observed with cells cultivated on the non-coated titanium alloy discs.

TABLE 1
Effect of gallium on the expression of the genes encoding for CA IX and for ALP.

| | | without Ga | | with Ga | |
|---|---|---|---|---|---|
| | | −MAC | +MAC | −MAC | +MAC |
| Incubation | Gene | 0 d | 5 d | 0 d | 5 d |
| Without | CA IX | 0.27 ± 0.02 | 0.81 ± 0.08 | 0.24 ± 0.05 | 0.89 ± 0.11 |
| discs | | | | | |
| Titanium | CA IX | 0.31 ± 0.03 | nd | 0.28 ± 0.05 | nd |
| alloy discs | | | | | |
| Ti—Ca—polyP | CA IX | 0.25 ± 0.03 | 0.59 ± 0.06 | 0.27 ± 0.04 | 0.96 ± 0.15 |
| discs | | | | | |
| Without | ALP | 0.029 ± 0.004 | 0.097 ± 0.007 | 0.027 ± 0.004 | 0.111 ± 0.010 |
| discs | | | | | |
| Titanium | ALP | 0.028 ± 0.003 | nd | 0.030 ± 0.002 | nd |
| alloy discs | | | | | |
| Ti—Ca—polyP | ALP | 0.030 ± 0.003 | 0.074 ± 0.007 | 0.027 ± 0.003 | 0.115 ± 0.007 |
| discs | | | | | |

The experiment was performed as described in the legend to FIG. 8, in the absence or in the presence of 100 μM gallium nitrate in the assay mixture.
The expression values were normalized to the expression of GAPDH.
The cells were cultivated either without any titanium discs, or either onto titanium alloy discs or on Ti—Ca—polyP discs.
The cultures were incubated at first in the absence of the MAC for 3 d and then transferred to medium, supplemented with the MAC, and the incubation was continued for additional 3 or 5 d.
nd, not detectable.

### XRD Analyses

The phase identification of the “HA” as well as the polyP-HA particles was performed by applying the powder X-ray diffraction (XRD) method (FIG. 10). While for pure Na-polyP no distinct diffraction signals can be resolved, indicating an amorphous phase, pure “HA” as well as “HA(2.5)polyP” and “HA(5)polyP” exhibit broad diffraction peaks indicating formation of HA with low crystallinity; no other crystalline phase was detected (JCPDS [http://www.icdd.com/] #09-0432). However, when the amount of polyP increases to 10 wt. %, as in “aCaP(10)polyP”, no signs of crystallinity are seen in the XRD pattern (FIG. 10). These results show that the degree of crystallinity of the prepared HA sample progressively decreases with the increase in polyP content.

### FTIR Spectral Analysis

All the spectra for CaP recorded here, like pure “HA”, as well as “HA(2.5)polyP” and “HA(5)polyP”, showed the typical HA bandings (FIGS. 11A and B), except for “aCaP(10)polyP”. As expected, the typical absorption bands of HA with high intensity are recorded at 1090 cm⁻¹, 1015 cm⁻¹ and 960 cm⁻¹, with the symmetric ν₁ (PO₄³⁻) and the asymmetric ν₃ (PO₄³⁻) stretching vibrations. In addition, at ν₄ (PO₄³⁻) the peaks at 556 cm⁻¹ and 604 cm⁻¹ are characteristic bending vibrations. In contrast, the spectrum for “aCaP(10)polyP” shows a distinct shift of the phosphate absorption band for the symmetric ν₁ (PO₄³⁻) and asymmetric ν₃ (PO₄³⁻) stretching vibrations in the region between 1100-900 cm⁻¹ and also the ν₄ (PO₄³⁻) harmonics of P═O bending vibrations, which appeared as one peak centered around 610 cm⁻¹. In addition, a wide absorption band within the range from ˜3600 cm⁻¹ up to 3100 cm points on ν₃ and ν₁ with H₂O molecules bonded with hydrogen for stretching modes. The absorption band at 1629 cm⁻¹ is attributed to the deformation mode ν₂ of H₂O molecules, proving the presence of physically adsorbed water in the synthesized samples. It has been reported that the vibration bands around 556 cm⁻¹ and 604 cm⁻¹ in the FTIR spectra of CaP reflect the characteristic bending signals of the harmonic vibration for crystalline PO₄³⁻; shifting of the two peaks indicate the transformation from crystalline to amorphous phase. This shift is clearly seen in the pattern of “aCaP(10)polyP”, where the two peaks now show up as one peak, indicating the amorphous nature of this sample. This finding is also in agreement with the reported XRD pattern (FIG. 10).

For comparison, the spectrum of polyP is also included in the CaP tracings (FIG. 11). It is apparent that for polyP a peak near 1261 cm⁻¹ appears that is assigned to the asymmetric stretching mode of (O—P═O), characteristics for polyP. The absorption bands close to 1090 cm⁻¹ and 960 cm⁻¹ are assigned to the asymmetric and symmetric stretching modes of (O—P—O), respectively. These signals further confirm the presence of polyP. In addition, the absorption band near 864 cm⁻¹ is indicative for the asymmetric stretching modes of the P—O—P linkages and the partially split band centered around 763 cm⁻¹ should be attributed to the symmetric stretching modes of these linkages.

### TEM and Particle Size Distribution Results

The morphologies of the CaP samples were analyzed by TEM. The “HA” sample showed needle-like nano-crystals with an average length of 39±8 nm and a width of 14±4 nm (FIG. 12A). Almost the same dimensions were visualized in “HA(2.5)polyP” samples with a length of 42±10 nm and a width of 9±5 nm (FIG. 12B). Slightly longer are the crystals present in the “HA(5)polyP” preparation with 56±12 and 6±3 nm in width (FIG. 12C). In contrast, the CaP preparation, containing the highest proportion of polyP, “aCaP(10)polyP”, showed particles with different morphologies (FIG. 12D). Instead of needle-like structure spherical particles with a diameter of 70 to 120 nm (96±15 nm) are resolved. Those particles have the tendency to agglomerate to larger entities.

### Effect of CaP Samples and of Ca-polyP Nanoparticles on Cell Growth

The cell viability and growth of SaOS-2 cells onto the CaP samples were tested by applying the MTT assay. Those samples were added at a concentration of 100 μg/mL to the cells. In parallel, an incubation was performed with 10 μg/mL of Ca-polyP nanoparticles, “aCa-polyP-NP”, a sample which has been proven to increase the growth rate of the cells and to cause an increased gene expression of ALP and COL-I.

The results revealed that, after an incubation period of 2 d no significant differences in the growth of the cells on the different substrates are seen. However, after an incubation period of 3 d a significant increase in the growth of the SaOS-2 cells in the presence of “aCa-polyP-NP” (from 1.74±0.19 [day 2] to 2.45±0.20 absorbance units) is measured. The increase of the growth onto the different CaP samples is lower and significant for “HA” (from 1.54±0.18 to 1.93±0.21) and for “aCaP(10)polyP” (from 1.54±0.18 to 2.31±0.25).

### SEM Analyses

Cells were cultivated onto either pure “HA” or onto “aCaP(10)polyP” discs for 3 d. Then the samples were fixed with paraformaldehyde and inspected by SEM. It is seen that in both assays the cells firmly attach to the substrate both for the “HA” and the “aCaP(10)polyP” cultures. At higher magnification the property of the cells for spreading becomes obvious.

### Gene Expression Propensity of SaOS-2 Cells on CaP

The bone-related SaOS-2 cells were cultivated initially for 3 d and then transferred into new medium, lacking or supplemented with MAC and containing also the CaP samples (100 μg/mL) or the polyP nanoparticles (10 μg/mL). Then the incubation was continued for 7 d prior to qRT-PCR analyses to determine the steady-state level of transcripts for COL-I or ALP (FIG. 13).

The determinations revealed that the expression of COL-I at the time of seeding the cells is low with 0.26±0.07 expression units, related to the expression of GAPDH. In the absence of MAC and the 7 d presence of the CaP samples or the polyP nanoparticles the expression level significantly increased after incubation with “HA(2.5)polyP” (to 0.35±0.05), “HA(5)polyP” (to 0.43±0.05), as well as “aCaP(10)polyP” (to 0.52±0.06), and, as expected, also for polyP “aCa-polyP-NP” (to 0.63±0.07); FIG. 13A. After exposure to MAC all steady-state expression levels are significantly higher and reach values, e.g., of 0.41±0.05 for “HA”, and of 1.06±0.11 for “aCaP(10)polyP”. The latter value for “aCaP(10)polyP” is close to the induction level which is seen in cells exposed to polyP nanoparticles with 1.38±0.16.

A comparable inducing expression pattern is recorded for the ALP gene, if correlated to the reference gene GAPDH. Again, in the absence of MAC the ALP expression level is lower compared to the values measured for cells incubated for 7 d in the presence of MAC (FIG. 13B). Only in the assays without the MAC but supplemented with polyP nanoparticles the increase of the expression level of ALP is significant (from 0.12±0.02 to 0.17±0.01). However, after exposure to MAC the expression levels for all polyP-containing CaP-preparations are significantly higher than the one seen during the seeding of the cells. The increased value for “HA(2.5)polyP” is 0.15±0.03, for “HA(5)polyP” 0.28±0.03, and for “aCaP(10)polyP” 0.89±0.09. Again the latter expression level is closer to the value determined for the polyP-exposed cells with 1.37±0.16, if compared to the samples containing smaller amounts of polyP.

Effect of polyP on Calcite Formation: FTIR and XRD Spectra

For all CaCO₃ solids the following FTIR signals were recorded: ν₁ (symmetric stretching) at ≈1080 cm⁻¹; ν₂ (out of-plane bending) at ≈870 cm⁻¹; ν₃ (doubly degenerate planar asymmetric stretching) at ≈1400 cm⁻¹ and ν₄ (doubly degenerate planar bending) at 700 cm⁻¹. The published IR data (Rodriguez-Blanco J D, Shaw S and Benning L G (2011) The kinetics and mechanisms of amorphous calcium carbonate (ACC) crystallization to calcite, via vaterite. Nanoscale 3:265-271) which were obtained with FTIR/KBr pellets, include peaks located at around 1400 cm⁻¹ (ν3), 876 cm⁻¹ (ν₂), and 714 cm⁻¹ (ν₄) for calcite and 1090 cm⁻¹ (ν₁), 870 cm⁻¹ (ν₂), and 745 cm⁻¹ (ν₄) for vaterite (FIG. 14). Our samples prepared in the absence of polyP are characterized as follows. For calcite the typical vibration bands 1391, 872 and 712 cm⁻¹ were recorded, while the samples prepared in presence of polyP showed the adsorption peaks at 1398, 869 and 742 cm⁻¹ for “CCP5” polyP as well as the bands at 1398, 869 and 741 cm⁻¹ for “CCP10” proving the formation of vaterite. It is apparent that the strength of the signal for vaterite around 741 cm⁻¹ decreases at higher content of polyP in the fabricated CaCO₃ solids, “CCP10” versus “CCP5”. This is indicative for the formation of ACC. Beside of the CO₃²⁻ absorption peaks, the peaks from 1200 cm⁻¹ to 950 cm⁻¹ correspond to the absorption peaks of phosphate in polyP.

The above result was confirmed with XRD in which the diffraction peaks of the sample prepared in absence of polyP, at approximately 23°, 30°, 36° and 40°, is given; those signals correspond to calcite. In contrast, the samples prepared in the presence of polyP (“CCP5”) showed peaks at approximately 24°, 27°, 32° and 44°, which also reflect the existence of vaterite. Furthermore, these data prove that the CaCO₃ solids, prepared in the absence of polyP were pure calcite (FIG. 15A), while the “CCP5” samples were composed of vaterite in association with ACC, as can be deduced from the low intensities of the signals and also the broadening of the diffraction peaks for sample “CCP5” (FIG. 15B). In consequence, the increase of the amount of polyP, as in “CCP10”, decreases the rate of transformation of ACC to vaterite. This is evident from the XRD pattern of “CCP10” sample which exhibits the amorphous nature of the sample, but also containing small amounts of vaterite.

### Morphology of the Solids Formed

The solids formed by precipitation from CaCl₂.2H₂O and Na₂CO₃ were studied by SEM. The photomicrographs of the particles, formed in the absence of polyP, show the typical crystalline calcite, the rhombohedral crystals surrounded by {104} faces; FIGS. 16A and B. The size of the particles varies between 5.3 to 8.9±2.4 μm. In contrast, those solids formed from CaCl₂.2H₂O and Na₂CO₃ in the presence of polyP show a different morphology. At the lower polyP concentration, the “CCP5” particles show a spherical appearance with an average size of the spherical crystals of 9.4±3.7 μm (FIGS. 16C and D); we attribute these particles to vaterite. They are surrounded by very abundantly accumulating small nanoparticles with a size range of 100 to 200 nm, which we assigned as ACC. Increasing the polyP, as in “CCP10”, the globular particles disappear and are replaced by penta/hexagonal flake shaped particles, 5-10 μm sized vaterite (FIGS. 16E and F).

### Effect of CaCO₃ Samples on Cell Growth/Viability

The cell growth/viability of SaOS-2 cells after exposure to the CaCO₃ preparations was determined by applying of the MTT assay (see above). The CaCO₃ samples were added at a concentration of 50 μg/mL to the cells. In parallel, a control assay lacking any CaCO₃ solids was performed. The results revealed that the increase in cell growth/viability from 0.70±0.11 at time 0 to approximately 1.1 absorbance units after 2 d and 2.35 units after 3 days changes only non-significantly among the control assays and the three CaCO₃ series (“CCP5”, “CCP10” or calcite).

### Stability of the CaCO₃ Solids in the Culture Medium

SaOS-2 cells grow in an adherent manner (Pautke C, et al (2004) Characterization of osteosarcoma cell lines MG-63, Saos-2 and U-2 OS in comparison to human osteoblasts. Anticancer Res 24:3743-3748). If the cultures are exposed to either calcite or “CCP5” solids the growth behavior onto the surfaces of the culture dishes is similar in assays containing either “CCP10” (FIGS. 17A and B) or calcite (FIGS. 17C and D). After 3 d the cells grow almost to confluency. However, it is remarkable that the number of mineral particles, floating in the culture medium, after this period of time, is strongly reduced in the assays containing “CCP10”, compared to those seen in calcite assays. This observation can be taken as an indication that the “CCP10” particles undergo dissolution during the 5 d incubation period. This finding is supported by the determination revealing that after 3 d incubation period in simulated body fluids (Oyane A, Kim H M, Furuya T, Kokubo T, Miyazaki T, Nakamura T (2003) Preparation and assessment of revised simulated body fluids. J Biomed Mater Res A 65:188-195) the amount of calcite particles decreases only by 5-10%, while only 35% of the “CCP10” particles can be recovered, as measured on the basis of sedimentable carbonate (data not shown).

Release of Ca²⁺ from the CaCO₃ Particles

In separate assays either calcite or “CCP10” was added into an 1 mL assay buffered with 1 M Tris-HCl (pH 7.4). While almost no Ca²⁺ is released from the calcite sample, already 6.8±1.1 μg/ml (68% of the total Ca²⁺ in the reaction mixture) was released from the “CCP10” after a period of 48 hr; this extent increases further during the total 192 hr of incubation (FIG. 18).

### Expression of ALP in SaOS-2 Cells as Well as in MSCs

The morphogenetic activity of the CaCO₃ samples towards SaOS-2 cells as well as the MSCs was determined in the absence and presence of MAC. Using SaOS-2 cells it was determined that in the absence of MAC the expression ratio between the ALP and the reference gene expression (GAPDH) significantly increases from 0.31±0.9 to ≈0.6. Within the sets of experiments without the MAC no significant differences are measured, irrespectively of the absence (control) or presence of the CaCO₃ samples in the assays (FIG. 19A). However, if the expression ratio (ALP:GAPDH) is determined in MAC activated cells then a significant increase of the ratio to 0.87±0.12 (in the control), to 1.74±0.23 (“CCP5”) or to 1.86±0.29 (“CCP10”) is measured. In contrast, no response of the cells in assays with calcite is measured (0.14±0.05).

A similar expression pattern of the ALP, if correlated to the reference GAPDH gene expression, is found if MSCs are used for the experiments. Again, in the presence of the MAC a significant increase of the expression ratio is seen assays in the absence of any CaCO₃ solid, as well as in the presence of both “CCP5” and “CCP10”. No inducing effect is determined in cells exposed to calcite (FIG. 19B).

### Expression of BMP2 in SaOS-2 Cells

The expression level of BMP2 in response to “CCP10” and polyP (Ca²⁺ complex) was determined by qRT-PCR analysis. SaOS-2 cells were incubated in mineralization medium (McCoy's medium/MAC) for up to 7 days. “CCP10” (50 μg/ml), polyP (Ca²⁺ complex; 5 μg/ml; corresponding to 50 μM with respect to phosphate) or calcite (50 μg/ml) were added to the cultures at the beginning of the experiments. After termination RNA was extracted from the cultures and subjected to qRT-PCR. The expression of the housekeeping gene GAPDH was used as reference. As shown in FIG. 20 the expression levels of BMP2 significantly increased 3 to 7 days after addition of “CCP10” or polyP (Ca²⁺ complex). However, the increase in BMBP2 expression was much faster for “CCP10” compared with polyP (Ca²⁺ complex). Maximum levels of BMB2 gene expression were already achieved after an incubation period of 3 days for “CC10P”, while the expression of this gene induced by polyP (Ca²⁺ complex) reached maximum levels (and similar levels compared with “CCP10”) only after a longer, 5 day incubation period. At day 3 the expression level of BMP2 in response to “CCP10” was significantly (about 2-fold) higher compared with polyP (Ca²⁺ complex), indicating a “synergistic” effect of both components. After 7 days, the expression levels decreased for both “CCP10” and polyP (Ca²⁺ complex) but remained still significant. Calcite that is formed from metastable ACC in the absence of polyP did not show any stimulatory effect on BMP2 gene expression.

### Microspheres, Used for the Animal Studies

The control spheres, the “cont-mic” had a size of (≈845 μm [820±60 μm]; n=50), while those containing polyP were insignificantly slightly smaller (≈838 μm [816±65 μm]); FIGS. 21A and B. The texture of the microspheres surfaces was porous and had pores of 25-30 nm (not shown here). The content of polyP in the “polyP-mic” was 7.26±0.92%. The hardness of the particles was determined for both the “cont-mic” and the “polyP-mic”; the median RedYM stiffness of 26.99±6.22 kPa for the “cont-mic” and 23.96±23.96 kPa for the “polyP-mic” microspheres.

### Compatibility Studies in Rats

The microsphere samples (20 mg), both “cont-mic” and “polyP-mic” were inserted in the muscles of the back of rats, as described under “Materials and Methods”. After 2, 4, or 8 weeks tissue samples with the microspheres were removed, sliced and stained with hematoxylin solution. In none of the excised specimens any sign for a histopathological alteration could be seen in all of the three sacrificed laboratory animals per group both for the “cont-mic” and the “polyP-mic” series. After 2 weeks the regions, where the microspheres had been placed into the muscle, a few cells are scattered within the microsphere areas. However, after a 4 and 8 weeks stay of the “cont-mic” microspheres in the muscle area they appear to be empty or close to be cell-free. In contrast, within the “polyP-mic” microspheres already after 4 weeks an accumulation of the cells within the spheres are evident. After 8 weeks the spheres are almost filled with infiltrating cells.

Determinations of the hardness of the implant region after 8 weeks revealed a significant increase of the median RedYM stiffness of 33.13±7.97 kPa for the “cont-mic” and 60.11±12.13 kPa for the “polyP-mic” microspheres. The muscles of the back of rats before implantation have a median RedYM stiffness of 74.40±14.33 kPa.

### Methods

### Polyphosphate

The sodium polyphosphate (Na-polyP of an average chain of 40 phosphate units) used in the Examples has been obtained from Chemische Fabrik Budenheim (Budenheim; Germany).

### Ti—Ca-polyP Discs

Titanium alloy (Ti-6Al-4V) disks (15 mm in diameter and 2 mm in thickness, can be obtained, for example, from Nobel Biocare. Prior to use they are polished with emery paper (silicon carbide; Matador) followed by ultrasonic cleaning in distilled water, and subsequently washing in acetone (10 min) and in 40% ethyl alcohol solution (15 min), and finally rinsing in distilled water for 20 min. The samples are dried at 50° C. for 24 h. Then titanium alloy discs are incubated in 20 mL of 5 M HCl at room temperature for 6 h. After gentle washing in distilled water the discs were dried at room temperature and the treated disc samples were overlayed with 10 ml Ca-polyP nanoparticle suspension in the presence of the silane coupling agent (3-aminopropyl)trimethoxysilane [APTMS] (e.g., from Sigma-Aldrich).

Ca-polyP microparticles are prepared by mixing of 0.5 g of Na-polyP with ATPMS solution (1 wt %) in 100 ml water; then 0.1 g Ca²⁺-chloride dihydrate (CaCl₂.2H₂O) was added. The titanium disks were incubated in the above suspension for 3 h at a 90° C.; under those conditions a colloidal suspension was initially formed. The pH of the environment was adjusted to 8.0 to allow binding of polyP to the silane-etched titanium discs via Ca²⁺ ionic bonds/bridging. The samples remained in this suspension for 1 d. The influence of two different ATMPS concentrations (1 mg/assay and 2 mg/assay, respectively) on the morphology of the coat formed onto the titanium surface was studied. Finally, the specimens, titanium-Ca-polyP (Ti—Ca-polyP)discs, were removed and dried at 100° C. (see FIG. 1).

In the experiments described under Examples, if not mentioned otherwise, discs prepared with the higher proportion of APTMS and then with Ca-polyP have been used.

Synthesis of HA and polyP-Hydroxyapatite

Hydroxyapatite (HA) nanoparticles can be synthesized by a wet chemical precipitation method from calcium chloride (CaCl₂) as Ca²⁺ source, and ammonium phosphate dibasic ((NH₄)₂HPO₄) as phosphate source. To precipitate stoichiometric HA (Ca₁₀(PO₄)₆(OH)₂; Ca/P ratio of 1.667), 100 mL of 0.3 M aqueous solution of (NH₄)₂HPO₄ is dropwise added to 100 mL 0.5 M aqueous solution of CaCl₂. The amount of reagents is calculated in order to obtain the Ca/P molar ratio for HA of 10:6. The pH of the reaction is maintained at 10 with the addition of sodium hydroxide solution.

In order to prepare polyP-substituted HA nanoparticles of various polyP content, the starting components (CaCl₂ and (NH₄)₂HPO₄) are additionally supplemented with 2.5, 5 or 10 wt. % of Na-polyP (referred to HA, or the respective CaP preparation) as follows. The respective amount of Na-polyP, 0.12 g [“HA(2.5)polyP”], 0.25 g [“HA(5)polyP”] or 0.50 g [“aCaP(10)polyP”], is added to the aqueous solution of (NH₄)₂HPO₄; then this solution is added to the dissolved CaCl₂. The pH is kept at 10. The resulting precipitates are left at room temperature for 24 h. Then the precipitates are filtered, washed 3-times with distilled water before being dried in a hot air oven at 60° C. for 24 h. The final powders are termed “HA”, “HA(2.5)polyP”, “HA(5)polyP” and “aCaP(10)polyP”.

Fabrication of the polyP Nanoparticles

For comparative functional/biological studies amorphous Ca-polyP nanoparticles can be prepared as described (Müller W E G, Tolba E, Schröder H C, Diehl-Seifert B and Wang X H. Retinol encapsulated into amorphous Ca²⁺ polyphosphate nanospheres acts synergistically in MC3T3-E1 cells. Eur J Pharm Biopharm 2015; 93:214-223). In brief, 2.8 g of CaCl₂ in 30 mL distilled water are added dropwise to 1 g of Na-polyP, dissolved in 50 mL distilled water at a pH of 10.0. The amorphous Ca-polyP nanoparticles formed are washed in water and then dried at 50° C.; the preparation is termed “aCa-polyP-NP”. The average diameter of the spherical particles is 96±28 nm and they have an amorphous state (Müller W E G, et al. A new polyphosphate calcium material with morphogenetic activity. Materials Letters 2015c; 148:163-166).

### Preparation of Ca-Carbonate Microparticles

Ca-carbonate (CaCO₃) is prepared by direct precipitation in aqueous solutions (at room temperature), using CaCl₂.2H₂O solution and Na₂CO₃ solution at equimolar concentration ratio between Ca²⁺ and CO₃² through rapid mixing; for a scheme, see FIG. 3.

To study the effect of polyP on precipitated CaCO₃ the solution of 20 ml of 0.1 M NaOH is supplemented with 0.05 g or 0.1 g of Na-polyP to which 1.05 g of Na₂CO₃ is added; subsequently this solution is diluted with 30 mL of deionized water. Then 50 mL water, containing 1.47 g CaCl₂.2H₂O, is added. By this, 5% [w/w] (addition of 0.05 g Na-polyP) and 10% [w/w] (0.1 g Na-polyP) of polyP, respectively, is added to the CaCO₃ precipitation assay. The suspensions obtained are filtrated, washed with acetone and dried at room temperature. The samples are termed “CCP5” (0.05 g Na-polyP per CaCO₃ precipitation assay) or “CCP10” (0.1 g).

### Durability of the Ca-polyP Coat

The stability and the durability of the Ca-polyP coat around the titanium discs can be quantified, for example, by determination of the Ca²⁺ release from the discs. The control discs, as well as the Ti—Ca-polyP discs are submersed in simulated body fluid (SBF) but omitting Ca²⁺ as component; the pH is adjusted to 8.0. The assay volume is 1 ml and incubation is performed at 37° C. The Ca²⁺ concentration is determined by applying the complexometric titration method; the reagent Eriochrome Black T is used (e.g., from Sigma-Aldrich). In the experiments described under Examples, the surface thickness of the polyP coat on one plane of the discs has been determined microscopically to be ≈5 μm. In turn, the total amount of Ca-polyP (density of ≈3 g/ml) on one plane of the discs had a value of ≈2.4 mg.

Where indicated under Examples, 5 μg of alkaline phosphatase (ALP) from bovine intestinal mucosa (e.g. from Sigma; ≥6,500 DEA units/mg protein) was added to the reaction mixture.

### Microscopic Analysis

The light microscopic inspection of the discs can be performed, for example, with a VHX-600 Digital Microscope from KEYENCE, equipped either with a VH-Z25 zoom lens (25× to 175× magnification) or a VH-Z-100 long-distance high-performance zoom lens (up to 1000× magnification). The surface roughness can be measured, for example, by using the KEYENCE VK-analyser software. For the scanning electron microscopic (SEM) analyses, for example, a HITACHI SU 8000 electron microscope (Hitachi High-Technologies Europe GmbH, Krefeld, Germany) can be employed.

### Electron Microscopy and Energy Dispersive X-Ray Spectroscopy

For the transmission electron microscopic (TEM) analyses, for example, the TemCam-F416 (4K×4K) CCD camera (TVIPS), operated on a Tecnai 12 transmission electron microscope (FEI) at an accelerating voltage of 120 kV, can be used. The equipment is connected with a particle size analyzer (ImageJ); in the experiments, described under Examples, 25-50 crystals/spheres have been evaluated.

Scanning electron microscopic (SEM) analyses can be performed, for example, with an SU 8000 instrument (Hitachi High-Technologies Europe), at low voltage (1 kV). For the studies described under Examples, the cells were grown in the 6-well plates onto CaP preparations that had been pressed to 1 mm thick discs, with a diameter of 34 mm, for 3 d. The cells, growing on the CaP substrates are fixed with 4% paraformaldehyde.

Energy dispersive X-ray (EDX) spectroscopy can be performed, for example, with an EDAX Genesis EDX System attached to a scanning electron microscope (Nova 600 Nanolab; FEI) operating at 10 kV with a collection time of 30-45 s. Areas of approximately 5 μm² are analyzed.

EDX mapping can be performed, for example, with the Hitachi SU 8000 microscope, carried out at low voltage (<1 kV, analysis of near-surface organic surfaces). The SEM is coupled with an XFlash 5010 detector, an X-ray detector that allows the simultaneous EDX-based elemental analyses. This combination of devices is used for higher-voltage (10 kV) analysis, during which the XFlash 5010 detector is used for element mapping of the surfaces of the deposits. The HyperMap database is used for interpretation.

### X-Ray Diffraction Analyses

The X-ray diffraction (XRD) experiments can be performed as described (Raynaud S, et al. Calcium phosphate apatites with variable Ca/P atomic ratio I. Synthesis, characterisation and thermal stability of powders. Biomaterials 2002; 23:1065-1072). The patterns of dried powders can be registered, for example, on a Philips PW 1820 diffractometer with Cu_{Kα} radiation (λ=1.5418 Å, 40 kV, 30 mA) in the range 2θ=5-65° (Δ2θ=0.02, Δt=5 s). The HA crystals can be identified as described (Lee D S H, Pai Y, Chang S. Effect of thermal treatment of the hydroxyapatite powders on the micropore and microstructure of porous biphasic calcium phosphate composite granules. J Biomat Nanobiotechnol 2013; 4: 114-118).

### Fourier Transformed Infrared Spectroscopy

The Fourier transformed infrared (FTIR) spectroscopic analyses can be performed by using micro-milled (agate mortar and pestle) mineral powder, for example, in an ATR-FTIR spectroscope/Varian 660-IR spectrometer (Agilent), equipped with a Golden Gate ATR unit (Specac). Each spectrum shown under Examples represents the average of 100 scans with a spectral resolution of 4 cm⁻¹ (typically 550-1800 cm⁻¹). Baseline correction, smoothing, and analysis of the spectra can be achieved, for example, with the Varian 660-IR software package 5.2.0 (Agilent). Graphical display and annotation of the spectra can be performed, for example, with Origin Pro (version 8.5.1; OriginLab).

Release of Ca²⁺ from the CaCO₃ Particles

In separate assays 100 μg/ml of either calcite or “CCP10” are added into an Eppendorf tube containing 1 mL of 1 M Tris-HCl (pH 7.4). After incubating at room temperature for 2 h, 2 d, 3 d and 8 d samples of 100 μl are taken, centrifuged and the supernatant analyzed for Ca²⁺ concentration. The determination can be performed, for example, with the photometric test kit (e.g., Millipore/Merck Chemicals; article no. 100858 “Calcium Cell Test”). The blank values are subtracted from the test assays.

### Cultivation of SaOS-2 Cells

Bone cell like SaOS-2 cells (human osteogenic sarcoma cells) are cultured in McCoy's medium (Biochrom-Seromed), supplemented with 2 mM L-glutamine, 10% or 15% heat-inactivated fetal calf serum (FCS), and 100 units/ml penicillin and 100 μg/ml streptomycin. The cells are incubated in 25-cm² flasks or in 6-well plates (surface area 9.46 cm²; e.g. from Orange Scientifique) in a humidified incubator at 37° C. Routinely, the cultures are started with 3×10⁴ or 1×10⁴ cells/well in a total volume of 3 ml. Where indicated, the cultures are first incubated for a period of 3 d in the absence the mineralization-activating cocktail (MAC), comprising 5 mM β-glycerophosphate, 50 mM ascorbic acid and 10 nM dexamethasone. Then the cultures are continued to be incubated for up to 7 d in the absence or presence of the MAC. The HA/polyP mineral samples (100 μg/mL [HA, CaP] or 10 μg/mL [“aCa-polyP-NP”]), are added to each well at the beginning of the experiments. Every third day the culture medium is replaced by fresh medium/serum and, where indicated, also with MAC. For the studies with the discs, 24-well plates (e.g., from Corning; diameter of each well 15.6 mm) are used into which the 15 mm large discs are inserted. The assays are performed with a total volume of 2 ml of cells/medium/FCS.

In a further series of experiments, shown under Examples, the assays have been performed in the presence of 100 μM gallium nitrate.

### Cell Proliferation/Cell Viability Assays

Cell proliferation/growth can be determined, for example, by the colorimetric method, based on the tetrazolium salt XTT, e.g., Cell Proliferation Kit II (Roche), or 3-[4,5-dimethyl thiazole-2-yl]-2,5-diphenyl tetrazolium (MTT; #M2128, Sigma) (Wang X H, et al. (2014) Modulation of the initial mineralization process of SaOS-2 cells by carbonic anhydrase activators and polyphosphate. Calcif Tissue Int 94:495-509).

### Human Mesenchymal Stem Cells

The expression of ALP is determined, in parallel to the one in SaOS-2 cells, with human mesenchymal stem cells (MSC). The cells are isolated and cultivated using established methods (Wang X H, et al. (2014) The marine sponge-derived inorganic polymers, biosilica and polyphosphate, as morphogenetically active matrices/scaffolds for differentiation of human multipotent stromal cells: Potential application in 3D printing and distraction osteogenesis. Marine Drugs 12, 1131-1147).

### Reverse Transcription-Quantitative Real-Time PCR Analyses

The quantitative real-time RT [reverse transcription]-PCR (qRT-PCR) technique is applied to determine the effect of the discs on the expression levels of the following genes in SaOS-2 cells. In brief, RNA was extracted from the cells and the PCR reaction is performed using the following primer pairs: carbonic anhydrase IX (CA IX; NM_001216 human) Fwd: 5′-ACATATCTGCACTCCTGCCCTC-3′ [nt₉₇₇ to nt₉₉₈] (SEQ ID NO. 1) and Rev: 5′-GCTTAGCACTCAGCATCACTGTC-3′ [nt₁₁₀₅ to nt₁₀₈₃] (SEQ ID NO. 2), alkaline phosphatase (ALP; NM_000478.4) Fwd: 5′-TGCAGTACGAGCTGAACAGGAACA-3′ [nt₁₁₄₁ to nt₁₁₆₄] (SEQ ID NO. 3) and Rev: 5′-TCCACCAAATGTGAAGACGTGGGA-3′ [nt₁₄₁₈ to nt₁₃₉₅] (SEQ ID NO. 4), type I collagen (Col I; NM_000088.3) Fwd: 5′-GACTGCCAAAGAAGCCTTGCC-3′ [nt₅₀₇₃ to nt₅₀₉₃] (SEQ ID NO: 5) and Rev: 5′-TTCCTGACTCTCCTCCGAACCC-3′ [nt₅₁₁₉₆ to nt₅₁₇₅] (SEQ ID NO: 6), and BMP2 (bone morphogenic protein 2; NM_001200.2) Fwd: 5′-ACCCTTTGTACGTGGACTTC-3′ [nt₁₆₈₁ to nt₁₇₀₀] (SEQ ID NO: 7) and Rev: 5′-GTGGAGTTCAGATGATCAGC-3′ [nt₁₇₈₅ to nt₁₈₀₄] (SEQ ID NO: 8). The glyceraldehyde 3-phosphate dehydrogenase (GAPDH) was used as a reference (NM_002046.5) Fwd: 5′-CCGTCTAGAAAAACCTGCC-3′ [nt₉₂₉ to nt₉₄₇] (SEQ ID NO. 9) and Rev: 5′-GCCAAATTCGTTGTCATACC-3′ [nt₁₁₄₅ to nt₁₁₂₆] (SEQ ID NO. 10). The PCR reactions can be performed, for example, in an iCycler (Bio-Rad), applying the respective iCycler software. After determinations of the Cₜ values the expression of the respective transcripts are calculated.

### Preparation of PLGA-Based Microspheres

The microspheres, used for the animal experiments are produced as described in details (Wang S F, et al. (2014) Bioactive and biodegradable silica biomaterial for bone regeneration. Bone: 67:292-304). The microspheres lacking CCP10 are fabricated with 4 ml of a PLGA/dichloromethane solution (volume ratio 1:5); they are termed “cont-mic” (PLGA: poly(D,L-lactide-co-glycolide); lactide:glycolide [75:25]; mol. wt. 66,000-107,000). For the fabrication of microspheres containing CaCO₃/polyP, “CCP10” microspheres (“polyP-mic”) are added to the PLGA/dichloromethane mixture at a concentration of 20%. The viscous reaction mixture is pressed through a syringe with an aperture of 0.8 mm. By this approach, microspheres with an average diameter of ≈820 μm are obtained.

The content of polyP in the microspheres is determined as described (Mahadevaiah M S, et al. (2007) A simple spectrophotometric determination of phosphate in sugarcane juices, water and detergent samples. E-Journal of Chemistry 4:467-473).

### Determination of the Mechanical Properties

The mechanical properties of the microspheres and of the muscle tissue of the implant region (regenerating zone) can be determined, for example, with a nanoindenter, equipped with a cantilever that has been fused to the top of a ferruled optical fiber (Wang S F, et al. (2014) Bioactive and biodegradable silica biomaterial for bone regeneration. Bone 67:292-304). Using this technique the reduced Young's modulus (RedYM) is quantified.

### Compatibility Studies In Vivo

In the experiments described under Examples, Wistar rats of (male) genders, weighting between 240 g and 290 g (age: two months) are used; 3 animals from each group are used. Diet and water are provided ad libitum during the total experimental period. Prior to surgery the animals are treated with Ciprofloxacins 10 ml/kg of body weight for antibiotic prophylaxis. Then the animals are narcotized with chlorpromazine/Ketamin via intramuscular injection. Following routine disinfection incisions of ≈1 cm are made in the right and left half, perpendicularly to the vertebral axis at the upper limbs level. Following skin incision, the muscle is incised and dissected to accommodate the microspheres. The microspheres (≈20 mg in a volume of 100 μL) are introduced into the muscle and stabilized there in the deeper layer (Vidya S., Parameswaran A., Sugumaran V G (1994) Comparative evaluation of tissue. Compatibility of three root canal. Sealants in Rattus norwegicus: A Histopathological study. Endodontology 6: 7-17). After a period of 2, 4, or 8 weeks the animals are sacrificed and the specimens with the surrounding tissue are dissected and sliced. The samples are inspected macroscopically for inflammation, infection and discoloration. The samples are fixed in formalin, sliced, stained with Mayer's hematoxylin and then analyzed by optical microscopy (e.g., with an Olympus AHBT3 microscope).

### Statistical Analysis

The results are statistically evaluated using paired Student's t-test.

1. A method selected from the group consisting of:
A) a method for the production of biologically active coatings of titanium alloys, comprising the following steps:
a) preparing Ca-polyP microparticles by mixing an aqueous solution of Na-polyP with an aqueous solution of calcium chloride dihydrate (CaCl₂.2H₂O) for several hours at an elevated temperature, under formation of a colloidal suspension;
b) coupling said Ca-polyP microparticle colloidal suspension to a titanium alloy scaffold using a silane coupling agent; and
c) adjusting the pH value of the suspension of b) to a slightly alkaline value to allow binding of polyP to the silane-functionalized metal scaffold via Ca²⁺ ionic bond formation;
B) a method for the preparation of biologically active amorphous polyphosphate-substituted calcium phosphate particles (“aCaP-polyP”) comprising the following steps:
a) adding an aqueous solution of a polyphosphate salt to an aqueous solution of a phosphate source;
b) adding the resulting solution to a dissolved calcium salt;
c) adjusting the pH to an alkaline value; and
d) collecting, washing, and drying the resulting precipitate formed; and
C) a method for the preparation of biologically active amorphous calcium carbonate (ACC)-polyphosphate microparticles, comprising the following steps:
a) preparing an aqueous solution of a polyphosphate salt in about 0.1 M sodium hydroxide;
b) adding about 0.5 mol/L of sodium carbonate to said solution;
c) diluting the resulting solution with about 1.5 volumes of deionized water;
d) mixing said solution with the same volume of an aqueous solution containing calcium chloride, so that an about equimolar concentration ratio between calcium ions and carbonate ions results;
e) washing with a lower alkyl ketone at about room temperature; and
f) filtering and drying a precipitate as formed.

2-3. (canceled)

4. The method according to claim 1, wherein, in the method of part A), said titanium alloy is Ti-6Al-4V.

5. The method according to claim 1, wherein, in the method of part A), said silane coupling agent is (3-aminopropyl)trimethoxysilane [APTMS].

6. The method according to claim 1, wherein, in the method of part C) the concentration of the polyphosphate salt in step a) is in the range of about 0.001 mol/L to about 1.0 mol/L, based on phosphate.

7. The method according to claim 6, wherein the concentration of the polyphosphate salt in step a) is about 0.025 mol/L or about 0.05 mol/L, based on phosphate.

8. The method according to claim 1, wherein the polyphosphate salt is sodium polyphosphate.

9. The method according to claim 1, wherein the chain length of the polyphosphate is about 3 to about 1000 phosphate units.

10. The method according to claim 2, wherein, in the method of part B), the amount of the polyphosphate salt is higher than 5 wt. % referred to the calcium phosphate preparation.

11. The method according to claim 2, wherein, in the method of part B), the calcium salt is calcium chloride (CaCl₂) and the phosphate source is ammonium phosphate dibasic [(NH₄)₂HPO₄)].

12. The method according to claim 1, wherein, in the method of part A), the calcium polyphosphate microparticles are characterized by a stoichiometric ratio between 0.1 to 1 and 50 to 1 of phosphate to calcium.

13. The method according to claim 12, wherein the calcium polyphosphate microparticles are characterized by a stoichiometric ratio of 7 to 1 of phosphate to calcium.

14. The method according to claim 1, wherein, in the method of part B), the amount of the calcium salt and the amount of the reagent serving as phosphate source is calculated in order to obtain the Ca/P molar ratio for the calcium phosphate of 10:6.

15. The method according to claim 1, wherein, in the method of part A), the average size of the calcium polyphosphate microparticles is about 0.1 to about 30 μm.

16. The method according to claim 1, wherein, in the method of part B), the average size of the polyphosphate-substituted calcium phosphate particles (“aCaP-polyP”) is in the range of about 20 to about 300 nm.

17. The method according to claim 1, further comprising, in the method of part A), the step of producing biologically active titanium alloy implants.

18. The method according to claim 1, further comprising the step of producing a biologically active implant material.

19. The method according to claim 18, further comprising the step of including at least one gallium salt into said implant.

20. The method according to claim 18, wherein said biologically active implant material is an artificial bone implant.

21. An implant prepared by the method according to claim 18.

22. Use, as an implant, of the coating as produced according to the method of part A) of claim 1.

23. The method according to claim 18, wherein the biologically active implant material is an artificial bone implant.

24. A stabilized amorphous calcium carbonate (ACC) composition produced by the method of part C) according to claim 1.

25. A method for providing a dietary supplement, treating calcium deficiency, and/or preventing or treating osteoporosis, wherein said method comprises administering a stabilized ACC composition according to claim 24.

26-27. (canceled)


SEQUENCE LISTING (10 sequences)

SEQ ID NO: 1 - DNA, 22 bp, HOMO SAPIENS
acatatctgcactcctgccctc

SEQ ID NO: 2 - DNA, 23 bp, HOMO SAPIENS
gcttagcactcagcatcactgtc

SEQ ID NO: 3 - DNA, 24 bp, HOMO SAPIENS
tgcagtacgagctgaacaggaaca

SEQ ID NO: 4 - DNA, 24 bp, HOMO SAPIENS
tccaccaaatgtgaagacgtggga

SEQ ID NO: 5 - DNA, 21 bp, HOMO SAPIENS
gactgccaaagaagccttgcc

SEQ ID NO: 6 - DNA, 22 bp, HOMO SAPIENS
ttcctgactctcctccgaaccc

SEQ ID NO: 7 - DNA, 20 bp, HOMO SAPIENS
accctttgtacgtggacttc

SEQ ID NO: 8 - DNA, 20 bp, HOMO SAPIENS
gtggagttcagatgatcagc

SEQ ID NO: 9 - DNA, 19 bp, HOMO SAPIENS
ccgtctagaaaaacctgcc

SEQ ID NO: 10 - DNA, 20 bp, HOMO SAPIENS
gccaaattcgttgtcatacc